(19) United States
(12) Patent Application Publication
(10) Pub. No.: US 2019/0345447 A1
(43) Pub. Date: Nov. 14, 2019

(54) DENDRITIC CELL PREPARATIONS, COMPOSITIONS THEREOF AND METHODS OF USING SAME

(71) Applicant: HADASIT MEDICAL RESEARCH SERVICES AND DEVELOPMENT LTD., Jerusalem (IL)

(72) Inventors: Uriel TRAHTEMBERG, Aminadav (IL); Dror MEVORACH, Jerusalem (IL)

(21) Appl. No.: 16/337,559

(22) PCT Filed: Sep. 28, 2017
(86) PCT No.: PCT/IL2017/051096

§ 371 Date: Mar. 28, 2019

Related U.S. Application Data
(60) Provisional application No. 62/401,250, filed on Sep. 29, 2016.

(51) Int. Cl. C12N 5/0784; A61K 39/00; A61K 35/15; C07K 16/28; A61P 35/02; C07K 14/705; C07K 14/725

(52) U.S. Cl. CPC C12N 5/0639; C07K 2317/31; A61K 39/001113; A61K 35/15; C07K 16/2803; A61P 35/02; C07K 14/70578; C07K 14/7051; C12N 2501/22; C12N 2501/2304; A61K 2039/5154; A61K 2039/5156; A61K 2039/5158; A61K 2039/804; C07K 2319/02; C07K 2319/03; C07K 2319/30; C07K 2319/33; A61K 39/001112

(57) ABSTRACT

The invention relates to cell preparations comprising dendritic cell (DC) sub-populations, methods of obtaining such cell preparations, and the use of such preparations for improved immune and cancer therapy. More specifically, embodiments of the invention relate to the production and use of substantially pure human DC subpopulations, useful in the preparation of vaccines against inflammatory diseases and cancer, as well as cell preparations for eliciting immuno-tolerance.

### FIELD OF THE INVENTION

The invention relates to preparations comprising defined dendritic cell sub-populations, methods of obtaining such cell preparations, and the use of such preparations for providing improved immunomodulation and cancer therapy.

### BACKGROUND OF THE INVENTION

Dendritic cells (DCs) are antigen-presenting cells of the mammalian immune system. Their main function is to process antigen material and present it on the cell surface to the T cells of the immune system. They act as messengers between the innate and the adaptive immune systems. Upon activation by external stimuli, DCs undergo a maturation process that encompasses structural, phenotypic and functional changes, which make them the most powerful initiators of adaptive immunity. DCs interact with all cells of the immune system, either directly or through secreted mediators, in both central lymphoid organs and at the immune periphery. DCs can mature in different routes, and their maturation via alternate processes can result in varied effector functions. For example, upon encountering tolerogenic stimuli, the DCs response ranges from indifference, to apoptosis, to acquisition of a tolerogenic phenotype and function that induces tolerance among other immune cells (Tisch et al. 2010). DCs response may or may not be accompanied by migration of the DCs.

DC subpopulations with different characteristics and functions have been previously identified and shown to perform varying roles. Subpopulations have commonly been defined based on their structural phenotype; however, this phenotype is only a surrogate, since it is their specific functions that are of interest for understanding and using DCs. Recent reviews have explored certain subpopulations in depth (Liu et al., 2010, Mildner et al., 2014, Schlitzer et al., 2014). Briefly, murine DCs found in the spleen and lymph nodes have been separated into CD8⁺ and CD8⁻ subtypes, which can be further subdivided. These organs also harbor migratory DCs that come from the periphery. The characteristics of non-lymphoid DCs also vary, with differing characteristics having been described for DCs in various tissues; the skin, gut, and lungs have been studied most frequently. These tissue DCs are commonly initially classified according to their CD11b expression, followed by tissue-specific markers. Distinct from these classical and tissue-resident DCs are plasmacytoid DCs, which specialize in antiviral responses. Finally, while the previously described DCs descend from bone marrow precursors, monocyte-derived dendritic cells (mdDCs) are derived from monocytes.

Knowledge of human DC subpopulations is not as well-developed in comparison with their murine counterparts (Schlitzer et al., 2014, Merad et al., 2013), and the gap between the understanding of mouse and human monocyte derived DCs, in particular, is significant (Mildner et al., 2013). In addition, collective understanding of the extent of correlation between observations of DCs, including mdDCs, in-vivo and those generated in-vitro, is far less well understood in humans than in mice (Collin et al., 2013, Boltjes et al., 2014). Still, the availability and plasticity of mdDCs make them a prime target for human research (Palucka et al., 2013).

Certain studies have described various mature human DCs, differing according to the protocols or sera used to produce them (Duperrier et al. 2000, Chang et al. 2000, Xia et al., 2002, Nunez Sondergaard et al., 2004). In mice, most DCs do not seem to arise from monocytes in the steady state (Liu et al. 2009). Indeed, monocytes have been shown to form DCs in inflammation (Geissmann et al. 2003), but also to reconstitute a portion of intestinal DCs following their ablation (Varol et al. 2009), and to be incorporated into different tissues as DCs in other studies (Geissmann et al. 2003, Dominguez et al., 2009); thus, the common conception that all murine mdDCs are inflammatory is called into question (Mildner et al. 2014). In addition, the understanding of human monocyte differentiation into DCs in-vivo remains a work in progress (Auffray et al. 2009, Alonso et al. 2011).

The uptake of dying cells is of great relevance for DC function, serving as an important means for DCs to obtain antigens and sample their environment in an ever-lasting process of peripheral tolerance (Hammer et al. 2013). Different modes of cell death are associated with signals that influence the DCs activation state (Green et al. 2009, Sancho et al., 2009). In mice, the CD8α⁺ subpopulation specializes in the uptake of dying cells and cross-presentation of their antigens. Human myeloid DCs that are positive for the surface markers BDCA3 (CD141) and CLEC9A are analogous to this subpopulation. These and other works have shown that the context of a cell's death and its interaction with an ingesting DC can strongly influence the final outcome that the DC itself will effect (Green et al. 2009).

However, little attention has been given in the literature to the death of the mdDCs' themselves. DCs have a major role in the stimulation of T and B cells for either activation or tolerization, and their lifespan is an important regulator of the duration of this stimulus. Common laboratory protocols for T cell expansion use irradiated, mitomycin C-treated, or fixed antigen-presenting cells (APCs), or even use fixed molecular platforms as an alternative for APC. Certain previous experiments used artificial APCs as vaccines. These examples show that injured or even inert APCs and APC-like constructs are functional. Therefore, the study of DC death characteristics is important, since even dying DCs could have immune effects. Immune cell patterns of death are an integral part of their function, as exemplified by the activation-induced death of T cells. Certain groups have shown that cells committed to die can actively produce immunomodulatory proteins de-novo (Stein et al. 2000, Krispin et al. 2006). In-vivo, DC's death can have different results depending on its state and location (Stranges et al. 2007). There are various and conflicting reports on DC death biology, especially on the role of Fas and the bcl-2 family (reviewed in Kushwah et al. 2010). Nevertheless, it is clear that DCs death is a regulated event that is affected by, and also affects, its state and environment.

WO 2014/087408, WO 2006/117786 and WO 2002/060376, listing some of the inventors of the present invention, relate to the production and/or use of apoptotic or necrotic cell preparations, including, inter alia, DCs or other immune cells.

Various methods for the generation and/or use of DC-based preparations and vaccines are described, for example, in WO 2016/145317, WO 2016/036319, US 2010/0105135, WO 2007/084105, US 2017/151281 and US 2004/0038398. US 2014/377761 discloses methods for determining if a dendritic cell belongs to a tolerogenic dendritic cell subset or to an effector dendritic cell subset, methods for determining if a patient undergoing immunotherapy, and/or who has been administered with a vaccine, is developing an immune response oriented either towards a regulatory T cell response or towards an effector T cell response, and methods of determining response to immunotherapy.

As described herein, several studies have disclosed or suggested the existence of different populations of mature mdDCs. However, in clinical practice, it is particularly desirable to obtain large quantities of immature DCs such as immature mdDCs (i-mdDCs), to be manipulated as desired for a variety of applications, e.g. in the production of the immunotherapy or vaccines. Current protocols used in research and in cell therapy lead to the production of heterogeneous i-mdDC preparations, typically comprising varying proportions of distinct cell populations that may exert opposing functions, thereby leading to reduced efficacy and potentially undesired effects. Thus, the production of homogenous i-mdDCs preparations, comprising substantially pure i-mdDC subpopulations, would be highly advantageous for clinical and research purposes alike.

### SUMMARY OF THE INVENTION

The invention relates to cell preparations comprising defined dendritic cell (DC) sub-populations, methods of obtaining such cell preparations, and the use of such preparations for, e.g. improved immune and cancer therapies. More specifically, embodiments of the invention relate to the production and use of specific human monocyte-derived DC (mdDC) subpopulations, useful in the preparation of e.g. vaccines against inflammatory diseases and cancer, or for eliciting or enhancing immune tolerance.

Human mdDCs are versatile immune cells that are used widely for research and experimental therapies. Although different culture conditions were shown to affect their characteristics at the mature stage, there are no known subpopulations of immature human mdDC (i-mdDCs). The invention is based, in part, on the unexpected experimental generation of two distinct mdDC subpopulations, herein designated small (DC-S) and large (DC-L) mdDC, isolated from human i-mdDCs generated ex-vivo. The two cell populations were found to exhibit differences in their phenotype, morphology, transcriptome, phagocytosis capability, activation, cell death, capability to uptake of dying cells, and response to dying cell uptake. In view of the unique characteristics and functions of these two cell populations, they were unexpectedly found to be useful for various applications, providing unexpectedly improved therapeutic modalities.

It has now been found that morphologically, DC-L (also referred to herein as DC-Large) are larger (size^{high}), more granular (gran^{high}) and have a more complex cell membrane (complexity^{high}) compared to DC-S (also referred to herein as DC-Small). Phenotypically, DC-L show higher expression of a wide panel of surface molecules and stronger responses to maturation stimuli compared to DC-S. Transcriptomic analysis revealed their separate identities and findings were consistent with the phenotypes observed. Although they show similar apoptotic cell uptake, DC-L have different capabilities for phagocytosis, demonstrate better antigen processing, and have significantly better necrotic cell uptake compared to the DC-S. These subpopulations also have different patterns of cell death, with DC-L presenting an inflammatory, “dangerous” phenotype while DC-S mostly downregulate their surface markers upon cell death. In addition, apoptotic cells induce an immune-suppressed phenotype, which becomes more pronounced among DC-L, especially after the addition of lipopolysaccharide, compared to DC-S.

Accordingly, the invention relates in some embodiments to cell preparations comprising substantially pure DC-S or DC-L populations (e.g. immature or mature DC-S or DC-L), to methods for producing such preparations, and to their use in e.g. the manufacture of cell vaccines and immunomodulatory therapies.

Thus, monocytes (e.g. human peripheral blood monocytes) may be differentiated according to exemplary embodiments of the invention into mdDC, from which substantially purified DC-S or DC-L may be obtained using cell sorting e.g. by flow cytometry. According to various embodiments, substantially purified cell populations can then be exposed to stimuli such as antigens, dying cells and/or other modulators (e.g. cytokines or other maturation signals), for the preparation of various immuno-modulating cell compositions, to be administered to a subject in need thereof. For example, in some embodiments, DC-L preparations may advantageously be used in the manufacture of cell vaccines, useful for the treatment or amelioration of cancer and infective diseases, and for the induction of immunogenic reactions towards antigens implicated in the etiology and/or pathology of such disorders. In contradistinction, DC-S preparations may advantageously be used in other exemplary embodiments for the manufacture of cell compositions useful for the treatment or amelioration of autoimmune and inflammatory diseases, and for the induction of tolerogenic immune reactions towards antigens implicated in the etiology and/or pathology of such diseases.

According to one aspect, the invention relates to preparations of substantially purified DC-S or DC-L populations (e.g. in their immature form, namely iDC-S or iDC-L, respectively).

In another aspect, the invention provides methods for generating preparations of substantially purified DC-S or DC-L populations.

In another aspect, the invention is directed to methods for preparing cell vaccines or immuno-modulating cell compositions comprising preparations of substantially purified DC-S or DC-L populations.

In another aspect, the invention is directed to cell vaccines or immuno-modulating cell compositions comprising preparations of substantially purified DC-S or DC-L populations.

In other aspects, the invention is directed to methods for preparing T cell therapies such as adoptive T cell immunotherapies, T cell vaccines and immuno-modulating T cell compositions comprising activation in the presence of preparations of substantially purified DC-S or DC-L populations, and to T cell therapies produced by these methods.

In yet another aspect, provided are methods for the treatment or amelioration of cancer and infective diseases.

In another aspect, the invention provides methods for inducing or enhancing an immunogenic reaction towards antigens implicated in the etiology and/or pathology of cancer and infective diseases.

In another aspect, the invention provides methods for the treatment or amelioration of autoimmune and inflammatory diseases.

In another aspect, the invention provides methods for inducing or enhancing a tolerogenic immune reaction towards antigens implicated in the etiology and/or pathology of autoimmune and inflammatory diseases. In another aspect, the invention provides methods for inducing T-cell suppression or anergy towards antigens implicated in the etiology and/or pathology of autoimmune and inflammatory diseases.

In another aspect, the invention is directed to methods for distinguishing between cell populations based on their morphology, phenotype and/or response to apoptotic stimuli.

The present invention provides, in one aspect, a cell preparation of a substantially pure human monocyte-derived dendritic cell (mdDC) population, selected from the group consisting of (a) DC-Large (DC-L), characterized, based on their mean size, granularity and membrane complexity, respectively, as size^{high}, gran^{high}, complexity^{high}; and (b) DC-Small (DC-S), characterized based on their mean size, granularity and membrane complexity, respectively as size^{low}, gran^{low}, complexity^{low}.

In certain embodiments, the human mdDC population is a population of immature mdDC cells. In certain embodiments, the human mdDC population is a population of mature mdDC cells.

In certain embodiments, the cells are immature DC-L (iDC-L), further characterized by their expression levels of surface markers as CD11c^{high}, CD47^{high}, and DCSIGN^{high}.

In certain embodiments, the cells are immature DC-S (iDC-S), further characterized by their expression levels of surface markers as CD11c^{low}, CD47^{low}, and DCSIGN^{low}.

In certain embodiments, the cells are mature DC-L (mDC-L), produced by incubating a population of iDC-L ex-vivo with at least one maturation signal. In certain embodiments, the maturation signal comprises lipopolysaccharide (LPS), zymosan, prostaglandin E2 (PgE2), tumor necrosis factor α (TNF-α), interleukin 1 β (IL-1β), transforming growth factor β (TGF-β), or combinations thereof. For example, the maturation signal may be selected from the group consisting of LPS; zymosan; a combination of PgE₂, TNF-α and IL-1β; TGF-β; and combinations thereof.

In certain embodiments, the cells are mature DC-S (mDC-S), produced by incubating a population of iDC-S ex-vivo with at least one maturation signal. In certain embodiments, the maturation signal comprises LPS, zymosan, PgE2, TNF-α, IL-1β, TGF-β, or combinations thereof. For example, the maturation signal may be selected from the group consisting of LPS; zymosan; a combination of PgE₂, TNF-α and IL-1β; TGF-β; and combinations thereof.

In certain embodiments, the cell population selected from the group consisting of DC-L and DC-S as described herein has been generated by a method comprising (a) providing a population of human mdDC by ex-vivo differentiation of monocytes in the presence of granulocyte-macrophage colony-stimulating factor (GM-CSF) and/or IL-4, and (b) isolating said cell population using cell sorting.

In certain embodiments, the cell sorting is based on at least one parameter selected from the group consisting of cell size, cell granularity, membrane complexity and the level of surface marker expression.

The present invention further provides, in another aspect, a cell vaccine or an immuno-modulating cell composition, comprising a cell preparation of a substantially pure human mdDC population, selected from the group consisting of DC-L and DC-S, and/or comprising a T cell preparation activated in the presence of said cell preparation. In various embodiments, the cell preparation is any one of the cell preparations as described above. In certain embodiments, the mdDC population has been genetically modified to express at least one targetor, co-stimulatory molecule and/or antigen. In certain embodiments, the at least one targetor comprises at least one chimeric antigen receptor (CAR).

In certain embodiments, the cell vaccine comprises a cell preparation of a substantially pure human mdDC population, selected from the group consisting of DC-L and DC-S as described above, pulsed with at least one disease-associated antigen, said cell vaccine further comprising a pharmaceutically acceptable carrier, excipient and/or adjuvant.

In certain embodiments, the human mdDC population is a population of mature DC-L, obtained by ex-vivo incubation of iDC-L in the presence of the at least one disease-associated antigen and at least one maturation signal. In certain embodiments, the disease-associated antigen is implicated in the etiology and/or pathology of cancer or an infective disease associated with a viral, bacterial fungal or parasitic infection. In certain embodiments, the disease-associated antigen is a tumor-associated antigen. In certain embodiments, the tumor-associated antigen is selected from the group consisting of B7H3, CAIX, CD44 v6/v7, CD171, CEA, EGFRvIII, EGP2, EGP40, EphA2, and ErbB2 (HER2).

In certain embodiments, the disease-associated antigen is a viral antigen. In certain embodiments, the viral antigen is associated with a Cytomegalovirus (CMV), Epstein Barr Virus (EBV), Human Immunodeficiency Virus (HIV), or influenza virus infection.

In certain embodiments, said mdDC population has been genetically modified to express at least one CAR that specifically binds a cell-surface tumor-associated antigen presented on a cancer cell. In certain embodiments, the cancer is selected from the group consisting of melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers. In certain embodiments, the cancer is acute lymphoid leukemia (ALL). In certain embodiments, the cell population expresses at least one CAR that specifically binds to CD19 and/or at least one CAR that specifically binds to CD22.

In certain embodiments, the immuno-modulating cell composition comprises a cell preparation of a substantially pure human mdDC population, selected from the group consisting of DC-L and DC-S as described above, pulsed with at least one disease-associated antigen implicated in the etiology and/or pathology of an autoimmune or inflammatory disease and/or with necrotic or apoptotic cells, said cell composition further comprising a pharmaceutically acceptable carrier, excipient and/or adjuvant.

In certain embodiments, the human mdDC population in said immuno-modulating cell composition is a population of mature DC-S obtained by ex-vivo incubation of iDC-S in the presence of the at least one antigen implicated in the etiology and/or pathology of an autoimmune or inflammatory disease and with at least one maturation signal.

In certain embodiments, the human mdDC population in said immuno-modulating cell composition is a population of mature DC-L obtained by ex-vivo incubation of iDC-L in the presence of necrotic or apoptotic cells and with at least one maturation signal.

In certain embodiments, the antigen is implicated in the etiology or pathology of a T cell mediated disease (e.g. autoimmune diseases, chronic non-resolving inflammatory diseases, and graft rejection).

In certain embodiments, the cell vaccine is for use in a method for the treatment or amelioration of cancer or an infective disease in a subject in need thereof.

In certain embodiments, the disease-associated antigen is a tumor-associated antigen, for use in a method of treating cancer in said subject. In certain embodiments, the disease-associated antigen is a viral antigen, for use in a method of treating a viral infection in said subject.

In certain embodiments, the cell vaccine is for use in a method for inducing or enhancing an immunogenic reaction towards antigens implicated in the etiology and/or pathology of cancer or an infective disease in a subject in need thereof. In certain embodiments, the antigen is a tumor-associated antigen. In certain embodiments, the tumor is selected from the group consisting of melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers. In certain embodiments, the disease-associated antigen is a viral antigen. In certain embodiments, the viral antigen is associated with a Cytomegalovirus (CMV), Epstein Barr Virus (EBV), Human Immunodeficiency Virus (HIV), or influenza virus infection.

In certain embodiments, the immuno-modulating cell composition is for use in a method for the treatment or amelioration of an autoimmune or inflammatory disease in a subject in need thereof.

In certain embodiments, the immuno-modulating cell composition is for use in a method for induction of a tolerogenic immune reaction towards antigens implicated in the etiology and/or pathology of an autoimmune or inflammatory disease in a subject in need thereof.

In certain embodiments, the antigen is implicated in the etiology or pathology of a T cell mediated disease selected from the group consisting of: autoimmune diseases, chronic non-resolving inflammatory diseases, and graft rejection. In certain embodiments, the autoimmune disease is selected from the group consisting of multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease and autoimmune hepatitis.

For example, without limitation, antigens related to autoimmune diseases (“auto-antigens”) include insulin and glutamic acid decarboxylase (GAD) and islet associated autoantigen in diabetes, myelin basic protein and proteolipid protein in multiple sclerosis, acetylcholine receptor in myasthenia gravis, and nuclear and ribosomal proteins, as well as nucleic acid protein complexes, such as histones, in lupus. Included among the autoantigens are further those derived from stem cells, or whole cell preparations from cell lines such as insulinoma, thymic tissue, B lymphoblastoid cells, or cells such as pancreatic beta cells which are generated from stem cells.

According to additional exemplary embodiments, autoantigens that may be used for preparing immune-modulating compositions for rheumatoid arteritis (RA) include but are not limited to type II bovine or chicken collagen, HCgp39, lyophilized Escherichia coli extract, the 15-mer synthetic peptide dnaJp1, and citrullinated proteins including but not limited to cit-vimentin, cit-fibrinogen, and cit-collagen type II, or peptides derived from these citrullinated proteins. Antigens useful for type-1 diabetes (T1D) include but are not limited to insulin, proinsulin, GAD65 (glutamic acid decarboxylase), IA-2 (islet antigen 2; tyrosine phosphatase), and the ZnT8 transporter (zinc transporter 8, localized on the membrane of insulin secretory granules), the immunomodulatory peptide DiaPep277 (derived from HSP60 protein), and other HSP60-derived peptides. Antigens useful for multiple sclerosis (MS) include but are not limited to myelin peptides including MBP13-32, MBP83-99, MBP111-129, MBP146-170, MOG1-20, MOG35-55, and PLP139-154.

The present invention further provides, in another aspect, an ex-vivo method for generating a cell preparation of a substantially pure human mdDC population selected from the group consisting of DC-L and DC-S as described above, comprising (a) providing a population of human mdDC by ex-vivo differentiation of monocytes in the presence of granulocyte-macrophage colony-stimulating factor (GM-CSF) and/or IL-4, and (b) isolating said cell population using cell sorting.

In certain embodiments, the cell sorting is based on at least one parameter selected from the group consisting of cell size, cell granularity, membrane complexity and the level of surface marker expression. In certain embodiments, the cell sorting is based on a plurality of parameters selected from the group consisting of cell size, cell granularity, membrane complexity and the level of surface marker expression, wherein each possibility represents a separate embodiment of the invention. In certain embodiments, the cell sorting is based on cell size, cell granularity, membrane complexity and the level of surface marker expression.

In certain embodiments, the surface marker comprises a plurality of markers selected from the group consisting of: αVβ5, CD11c, CD47, CD36, CD274 (PDL1), CD11b (CR3), CD6 (B7.2), CD85k (ILT3), CD40, CD324 (E cadherin), CD45, HLA-DR, TLR-1, CD33 (SIGLEC-3), CD266 (TWEAK-R), CD206, DCSIGN, CD200 (OX2), CD172a (SIRPα), CD273 (PDL2), CD141, CCR5, HLA-ABC, CD85j (ILT2), CD54 (ICAM-1), CD80 (B7.1), CD16 (FcγRIII), FcεRI, CD275 (ICOS-L), and CD25 (IL2R). In certain embodiments, the surface marker comprises a plurality of markers selected from the group consisting of: CD11c, CD47, and DCSIGN. In certain embodiments, the surface marker comprises CD11c, CD47, and DCSIGN.

In certain embodiments, steps (a) and (b) are performed without the addition of exogenous activation or maturation stimuli.

In certain embodiments, the ex-vivo method described above further comprises genetically modifying the mdDC population to express at least one CAR.

In certain embodiments, the ex-vivo method described above further comprises incubating the cells ex-vivo in the presence of the at least one disease-associated antigen and at least one maturation signal. In certain embodiments, the at least one maturation signal comprises LPS, zymosan, PgE2, TNF-α, IL-1β, TGF-β, or combinations thereof.

Other objects, features and advantages of the present invention will become clear from the following description and drawings.

### BRIEF DESCRIPTION OF THE DRAWINGS

FIG. 1A-1C. Light scatter and morphology of DC-S and DC-L. FIG. 1A) Forward vs side scatter dot plots of DCs analyzed by flow cytometry. Left panels show the ungated populations, right panels show the gating strategy used. The top panels show iDCs analyzed with FACScan, while the bottom panels show LPS-matured DCs analyzed in an LSR II. Gated populations represent viable cells (see main text). FIG. 1B) iDCs were prepared by cytocentrifugation, fixed with ethanol, and then stained with hematoxylin and eosin. In the top panel, two DCs with significant size differences are seen at high magnification. In the bottom panel, a lower magnification field shows a collection of DCs of different sizes. FIG. 1C) iDCs were sorted as described in Materials and Methods and then imaged live after addition of crystal violet using phase contrast. In the top panels there are two examples of DC-S, while the bottom panels show two examples of DC-L. Bar: 10 μm.

FIG. 2. Expression of surface markers on immature DC-L vs DC-S. The relative surface marker expression of DC-L vs DC-S at the immature stage is shown. DC-S median fluorescence intensity (MFI) was normalized to 100; values above and below 100 indicate higher and lower expression, respectively, of DC-L as compared to DC-S. *=p<0.05 for the DC-L/DC-S MFI ratio. n≥3 for all markers. Only SB- or PI-negative cells are shown. Error bars=±SEM. CCR2, CD1e, CD121b (IL1R2), CD163, HLA-G, LOX-1 (OLR1), OX40-L (CD252), RAGE, TIM-1, and TSLP-R were also tested; however, these surface markers were expressed at very low levels, precluding accurate quantification, or not expressed at all, thus, they are not shown.

FIG. 3A-3B. Changes in surface marker expression of DC-L vs DC-S following stimulation. The relative marker expression of DC-L vs DC-S at the immature stage (iDCs), as well as following stimulation with LPS, zymosan, a cytokine cocktail (CKC), or TGF-β is shown. The MFI of DC-S was normalized to 100; values above and below 100 indicate higher and lower expression, respectively, of DC-L as compared to DC-S. *=p<0.05 for the DC-L/DC-S MFI ratio. n≥3 for all markers. Only SB- or PI-negative cells shown. Error bars=±SEM.

FIG. 4A-4B. Characterization of differentially expressed transcripts in DC-S and DC-L. iDCs were sorted into DC-S and DC-L and re-plated for 24 hours with or without LPS, followed by RNA extraction. A pool of 3 experiments was analyzed using Affymetrix microarrays. Four pooled RNA datasets were obtained: DC-S at the immature stage and after LPS stimulation (iDC-S and mDC-S, respectively), and DC-L at the immature stage and after LPS stimulation (iDC-L and mDC-L, respectively). The data was preprocessed using Robust Multi-array Average (RMA) and a cutoff of 4 (log). In order to obtain the list of differentially expressed genes, the expression profiles of DC-S and DC-L were subtracted from each other. The list of genes presented in each category (iDC-S, iDC-L, mDC-S and mDC-L) represents genes that were differentially expressed, defined as a transcript with at least a twofold difference; thus, a gene that is present at similar levels in both subsets would be excluded from the results, even if highly expressed. Due to the cutoff used, fold changes indicate minimal overexpression (the differences can be larger but not smaller). A heat-map representation of the transcripts is shown at absolute levels after RMA and cutoff, in comparison to all the other samples. Black indicates high expression; light gray indicates low expression. Values were row-normalized; shown from top to bottom, from highest to lowest overexpression.

FIG. 5A-5E. Patterns of surface marker expression changes upon spontaneous DC death. DCs were labeled with fluorescent antibodies for marker expression and co-stained with SB. The cells were gated for DC-S and DC-L, as well as SB negative, low, and high, indicating advancing stages of spontaneous cell death during culture. FIG. 5A: Density plots of representative examples are shown. The MFI of each marker is indicated beside the gates. All gates include at least 50 events.

FIGS. 5B-5E (bar charts): DCs at the immature stage and after stimulation with LPS, CKC, and TGF-β, as indicated, were co-stained with fluorescent antibodies and SB, and gated as described above. FIG. 5B—CCR7, FIG. 5C—CD45, FIG. 5D—CD86, FIG. 5E—CD33. Values were normalized so that SB negative DC-S=100 (bold outline). n≥3 for all markers. *=p<0.05 for the DC-L/DC-S MFI ratio. ‡=p<0.05 for the DC-L/DC-S MFI ratio change vs SB negative (paired t-test). Error bars=±SEM.

FIG. 6. Imaging of live DCs stained with CD86 and PI. iDCs were labeled with CD86, co-stained with PI and imaged using an Amnis Imagestream™ cytometer. The cells were gated into DC-S (left column) and DC-L (right column), as well as PI-negative, low, and high, using an analogous scheme to the one used with other flow cytometers. Three representative examples from every set are shown.

FIG. 7A-7C. Phagocytosis, antigen-processing, and uptake of dying cells by DC-S vs DC-L. FIG. 7A) Targets were added to iDCs, to DCs previously stimulated for 24 hours with LPS, CKC, or TGF-β (“pre”), or simultaneously with LPS (“simul”), as indicated. *=p<0.05 for the DC-L/DC-S MFI ratio. n≥3. Only SB- or PI-negative cells shown. Error bars=±SEM. DCs were incubated with the indicated fluorescent targets for 8-12 hours and then analyzed by flow cytometry. FIG. 7B) Same as “A” but using DQ-ovalbumin, which is ovalbumin over-conjugated with fluorochrome, and thus self-quenching. After uptake and degradation, the fluorochromes in the resulting peptides are sparser and can fluoresce; therefore, higher fluorescence indicates higher uptake and/or processing of the original protein. FIG. 7C) DCs were incubated with DiD-labeled (fluorescent) apoptotic PMN at a ratio of 1:4 for 8-12 hours. Apoptotic cells were added to iDCs or to DCs previously stimulated for 24 hours with LPS or CKC, as indicated. Samples were then stained with HLA-DR or DCSIGN to specifically identify the DCs, and analyzed by flow cytometry. The MFI of DC-S was normalized to 100; values above and below 100 indicate higher and lower expression, respectively, among DC-L as compared to DC-S. *=p<0.05 for the DC-L/DC-S MFI ratio. †=p<0.05 for the DC-L/DC-S MFI ratio change in mature vs immature DCs (paired t-test). n≥3. Only SB- or PI-negative cells shown. Error bars=±SEM.

FIG. 8A-8B. Phenotype after interaction with apoptotic cells. DCs were mixed with apoptotic PBMC at a ratio of 1:4 for 24 hours. LPS was added 6 hours after the apoptotic cells, as indicated. Only SB- or PI-negative cells are shown; representative of 4 experiments. FIG. 8A: The change in the expression of surface markers for all DCs is shown, normalized for iDCs (bold outline). FIG. 8B: Same as the top panel, but instead of showing the results for all DCs, the MFI of iDC-S is normalized to 100; values above and below 100 indicate higher and lower expression, respectively, among DC-L as compared to DC-S.

FIG. 9. FSC vs SSC statistics. iDCs were gated according to the strategy described in Example 1 and FIG. 1. The statistics were compiled from 10 matching experiments. For the untreated, iDCs, DC-S are 54% in average, with a range of 46% to 69%, p=0.018 for the difference between DC-S and DC-L, n=10. After induction of maturation with LPS, the mean percentage of DC-S increases to an average of 61%, with a range of 53% to 71%, p<0.001 for the difference between DC-S and DC-L, n=10.

FIG. 10. Antibody specificity at all stages of cell death. iDCs were stained with a phycoerythrin-labeled, anti-CD91 antibody, with (right) or without (left) the presence of unlabeled antibody of the same clone. As can be seen, a reduction in the observed fluorescence of the same magnitude is seen for all stages of cell death (SB high, 76% reduction; SB low, 74% reduction; SB neg, 76% reduction). Similar results were obtained for HLA-DR and CD11c (n=3 for every antibody clone tested).

FIG. 11A-11B. Summarized patterns of surface marker change upon DC death. Heat-map representations of the different patterns of marker expression change upon cell death are shown, as detailed in FIGS. 5 and 6. Values were normalized so that SB/PI negative DC-S=100 (white squares). FIG. 11A: patterns 1 and 2, FIG. 11B: patterns 3 and 4. Values above and below 100 are represented in diagonal lines and dots, respectively.

FIG. 12A-12C. Production of CAR-T cells. FIG. 12A illustrates the structure of the Lenti-3^{rd} generation anti-CD19 CAR plasmid used in the experiment described in Example 7. The results of RT PCR tests to validate this structure are provided in FIG. 12B and in FIG. 12C.

### DETAILED DESCRIPTION OF THE INVENTION

The invention relates to cell preparations comprising dendritic cell (DC) sub-populations, methods of obtaining such cell preparations, and the use of such preparations for improved immune and cancer therapy. More specifically, embodiments of the invention relate to the production and use of substantially pure human monocyte-derived DC subpopulations, useful in the preparation of vaccines against inflammatory diseases and cancer, as well as cell preparations for eliciting immune tolerance.

Without being bound to any theory or mechanism, it has been surprisingly found that ex-vivo manipulation of monocyte-derived cells creates multiple DC populations of distinct morphology and substantially opposed immune functions. According to the principles of the present invention, these populations, especially when isolated and separated from each other, are useful in a variety of methods for manipulating immune processes ex-vivo and in-vivo. The present invention thus provides new DC-based tools for either increasing the immune response towards target cells such as cancer and virally-infected cells, or decreasing the immune response and increasing the tolerability towards antigens such as self-antigens.

Morphologically, the newly generated and isolated DC-L cells are larger (size^{high}), more granular (gran^{high} as determined by flow cytometry) and have a more complex cell membrane (complexity^{high}, as determined by microscopy, which may also be defined as bright^{low}) compared to the DC-S. Phenotypically, DC-L show higher expression of a wide panel of surface molecules and stronger responses to maturation stimuli compared to the DC-S. These discrete cell populations, as well as their respective subpopulations distinguished by their maturity level, may further be differentiated by various functional parameters, including transcriptomic gene expression, capabilities for phagocytosis, antigen processing, necrotic cell uptake, patterns of cell death, and response to uptake of apoptotic cells.

According to one aspect, the invention relates to preparations of substantially purified DC-S or DC-L populations (e.g. in their immature form, namely iDC-S or iDC-L, respectively).

In another aspect, the invention provides methods for generating preparations of substantially purified DC-S or DC-L populations.

In another aspect, the invention is directed to methods for preparing cell vaccines or immuno-modulating cell compositions comprising preparations of substantially purified DC-S or DC-L populations.

In another aspect, the invention is directed to cell vaccines or immuno-modulating cell compositions comprising preparations of substantially purified DC-S or DC-L populations.

In other aspects, the invention is directed to methods for preparing T cell therapies such as adoptive T cell immunotherapies, T cell vaccines and immuno-modulating T cell compositions comprising activation in the presence of preparations of substantially purified DC-S or DC-L populations, and to T cell therapies produced by these methods.

In yet another aspect, there are provided methods for the treatment or amelioration of cancer and infective diseases.

In another aspect, the invention provides methods for inducing or enhancing an immunogenic reaction towards antigens implicated in the etiology and/or pathology of cancer and infective diseases.

In another aspect, the invention provides methods for the treatment or amelioration of autoimmune and inflammatory diseases.

In another aspect, the invention provides methods for inducing or enhancing a tolerogenic immune reaction towards antigens implicated in the etiology and/or pathology of autoimmune and inflammatory diseases. In another aspect, the invention provides methods for inducing T-cell suppression or anergy towards antigens implicated in the etiology and/or pathology of autoimmune and inflammatory diseases.

In another aspect, the invention is directed to methods for distinguishing between cell populations based on their morphology, phenotype and/or response to apoptotic stimuli.

These and other embodiments of the invention will be described and exemplified further hereinbelow.

DC Preparations and Methods for their Generation

In another embodiment, the invention relates to preparations of substantially purified DC-S. In a particular embodiment, the invention relates to preparations of substantially purified iDC-S. In another embodiment, the invention relates to preparations of substantially purified DC-L. In a particular embodiment, invention relates to preparations of substantially purified iDC-L.

As used herein, the terms “substantially pure” or “substantially purified”, when used in connection with cell populations within a cell preparation, denote a purity level of at least 80%, at least 85%, at least 90%, at least 95%, at least 97% or at least 99% with respect to the existence of other cell populations. In particular, a substantially pure DC-L, DC-S, iDC-S, iDC-L, mDC-S or mDC-L preparation is substantially devoid (e.g. at a purity level disclosed herein) of other DC populations.

As used herein, the term “cell preparation” denotes an experimentally generated cell composition (e.g. by ex-vivo cell culture and separation methods as disclosed herein) of a particular cell type as disclosed herein.

The terms DC-L and DC-S refer to human mdDC subpopulations as described in the description and drawings herein. Both DC-S and DC-L express CD14 dimly and DCSIGN strongly, indicating that both are fully differentiated DCs. Both DC-S and DC-L express low levels of CCR7, CD83, and CD25, and both upregulate these and other maturation surface markers upon stimulation, confirming that there are two subpopulations that are initially immature. DC-L are characterized as gran^{high}, size^{high}, complexity^{high} human mdDC, whereas DC-S are characterized as gran^{low}, size^{low}, complexity^{low} human mdDC. The two subpopulations may also be differentiated based on their phenotype, transcriptome, phagocytosis, activation, cell death, uptake of dying cells, and/or response to dying cell uptake, substantially as described and exemplified herein. According to some embodiments, DC-S and DC-L may be isolated based on the aforementioned characteristics from human mdDC obtained by culturing in the presence of cytokines including, but not limited to GM-CSF and IL-4, e.g. substantially as described in further detailed below and further exemplified in the Examples section herein.

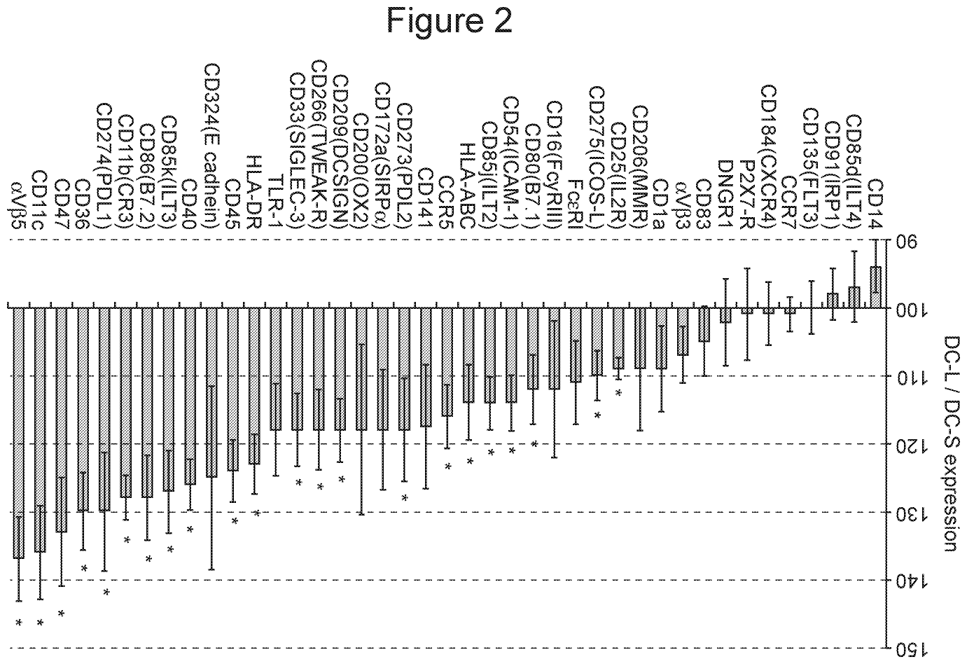

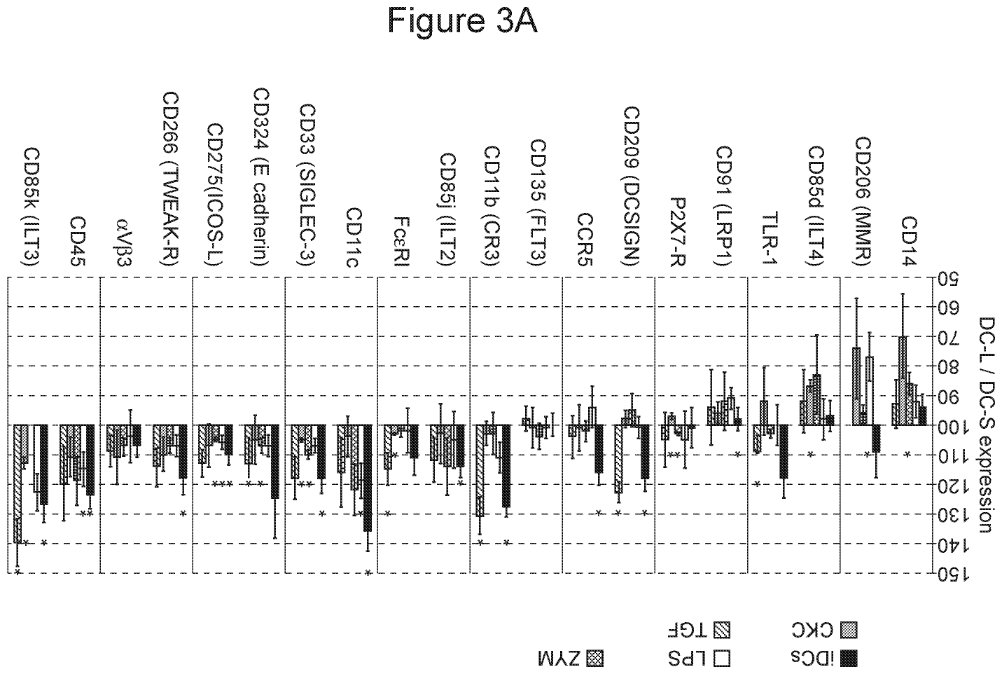

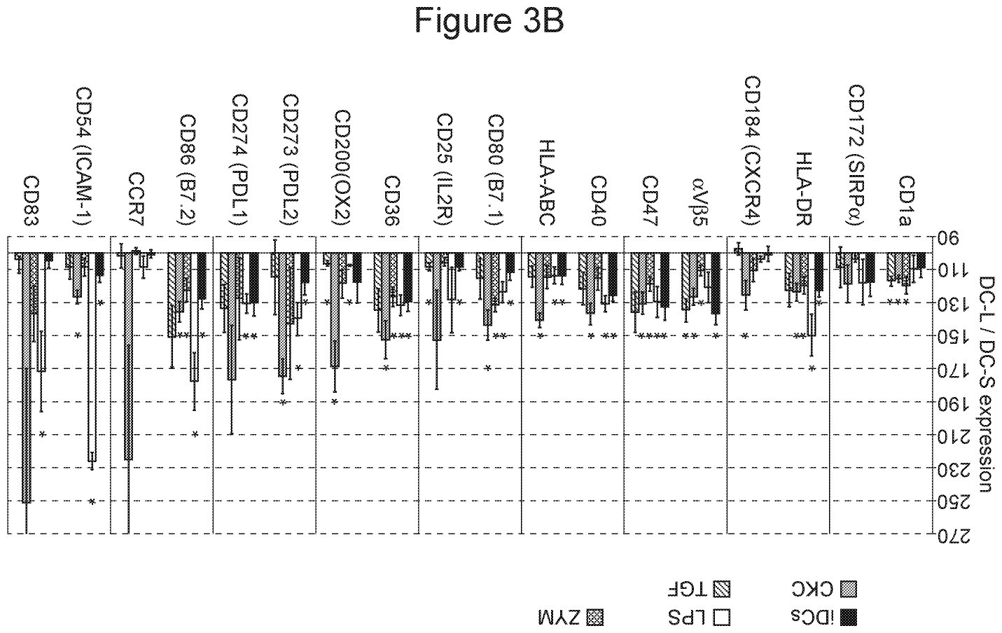

For example, DC-L and DC-S may be differentiated and/or isolated in various embodiments based on their expression levels of surface markers, as exemplified e.g. in FIGS. 2 and 3 herein. In some embodiments, DC-L may conveniently be further characterized at their immature stage as expressing strongly (“high”) a plurality of markers (e.g. at least 2, 3, 4, 5, 6 . . . 28 or 29) selected from the group consisting of: αVβ5, CD11c, CD47, CD36, CD274 (PDL1), CD11b (CR3), CD6 (B7.2), CD85k (ILT3), CD40, CD324 (E cadherin), CD45, HLA-DR, TLR-1, CD33 (SIGLEC-3), CD266 (TWEAK-R), CD206, DCSIGN, CD200 (OX2), CD172a (SIRPα), CD273 (PDL2), CD141, CCR5, HLA-ABC, CD85j (ILT2), CD54 (ICAM-1), CD80 (B7.1), CD16 (FcγRIII), FcεRI, CD275 (ICOS-L), and CD25 (IL2R), wherein each possibility represents a separate embodiment of the invention. In some embodiments, immature DC-L (iDC-L) are characterized as αVβ5^{high}, CD11c^{high}, CD47^{high}, CD36^{high}, CD274 (PDL1)^{high}, CD11b (CR3)^{high}, CD6 (B7.2)^{high}, CD85k (ILT3)^{high}, CD40^{high}, CD45^{high}, HLA-DR^{high} TLR-1^{high}, CD33 (SIGLEC_3)^{high}, CD266 (TWEAK-R)^{high}, CD206^{high}, DCSIGN^{high}, CD172a (SIRPα)^{high}, CD273 (PDL2)^{high}, CCR5^{high}, HLA-ABC^{high}, CD85j (ILT2)^{high}, CD54 (ICAM-1)^{high}, CD80 (B7.1)^{high}, CD275 (ICOS-L)^{high}, and CD25 (IL2R)^{high}. In other embodiments, iDC-L are characterized as CD11c^{high}, CD47^{high}, and DCSIGN^{high}.

In some embodiments, DC-S may conveniently be further characterized at their immature stage as expressing dimly (“low”) a plurality of markers (e.g. at least 2, 3, 4, 5, 6 . . . 28 or 29) selected from the group consisting of: αVβ5, CD11c, CD47, CD36, CD274 (PDL1), CD11b (CR3), CD6 (B7.2), CD85k (ILT3), CD40, CD324 (E cadherin), CD45, HLA-DR, TLR-1, CD33 (SIGLEC-3), CD266 (TWEAK-R), CD206, DCSIGN, CD200 (OX2), CD172a (SIRPα), CD273 (PDL2), CD141, CCR5, HLA-ABC, CD85j (ILT2), CD54 (ICAM-1), CD80 (B7.1), CD16 (FcγRIII), FCεRI, CD275 (ICOS-L), and CD25 (IL2R), wherein each possibility represents a separate embodiment of the invention. In some embodiments, immature DC-S (iDC-S) are characterized as αVβ5^{low}, CD11c^{low}, CD47^{low}, CD36^{low}, CD274 (PDL1)^{low}, CD11b (CR3)^{low}, CD6 (B7.2)^{low}, CD85k (ILT3)^{low}, CD40^{low}, CD45^{low}, HLA-DR^{low}, TLR-1^{low}, CD33 (SIGLEC-3)^{low}, CD266 (TWEAK-R)^{low}, CD206^{low}, DCSIGN^{low}, CD172a (SIRPα)^{low}, CD273 (PDL2)^{low}, CCR5^{low}, HLA-ABC^{low}, CD85j (ILT2)^{low}, CD54 (ICAM-1)^{low}, CD80 (B7.1)^{low}, CD275 (ICOS-L)^{low}, and CD25 (IL2R)^{low}. In other embodiments, iDC-S are characterized as CD11c^{low}, CD47^{low}, and DCSIGN^{low}.

The terms “positive” or “high”, “dim” or “low,” or “negative” for any of the cell-surface markers described herein, and all such designations are well accepted terms useful for the practice of the assays and methods described herein. A cell is considered “positive” or “high” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the assays and methods described herein, the cell must express the cell surface marker of interest on its surface. A cell is considered “dim” or “low” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell, but there exists another distinct population of cells that expresses the marker at a higher level, giving rise to at least two populations that are distinguishable when analyzed using, for example, flow cytometry. Similarly, a cell is considered “negative” for a cell-surface marker if it does not express the marker on its surface in amounts sufficient to be detected using methods known to those of skill in the art, such as contacting a cell with an antibody that binds specifically to that marker and subsequently performing flow cytometric analysis of such a contacted cell to determine whether the antibody is bound the cell.

Similarly, the terms “high” and “low”, are used herein in connection to physical properties of cells such as size and granularity according to their conventional scientifically accepted meaning. Accordingly, these terms refer to the identification and differentiation between of distinct sub-populations according to said parameters using methods known to those of skill in the art, such as flow cytometric analysis. The terms “high” and “low” may further be used herein in relation to a specific attribute of cells that may be detected qualitatively or quantitatively, dependent on the detection method. For example, additional detection methods may include microscopic evaluation, either with or without preceding staining.

For example, in regard to the complexity of a cell's membrane, the term “bright” as used in the labels bright^{high} and bright^{low} may be used interchangeably with “membrane complexity” e.g. as used in the labels complexity^{high} and complexity^{low}. A cell population identified as “complexity^{low}” refer to cells characterized by a membrane which has a substantially regular, circle (in 2D) or spherical (in 3D) shape. The label “complexity^{high}” refers to cells characterized by a membrane which has a substantially irregular and complex shape. Identification of complexity^{high} and complexity^{low} DC population is typically and conveniently determined by a skilled artisan using microscopic evaluation, e.g. light microscopy, electron microscopy or the like.

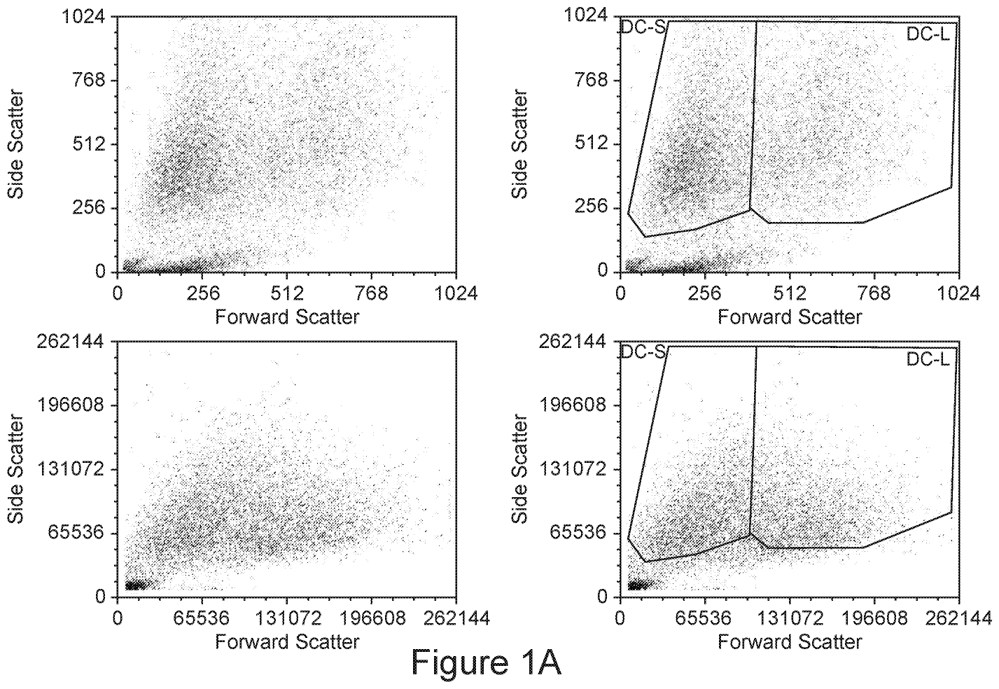

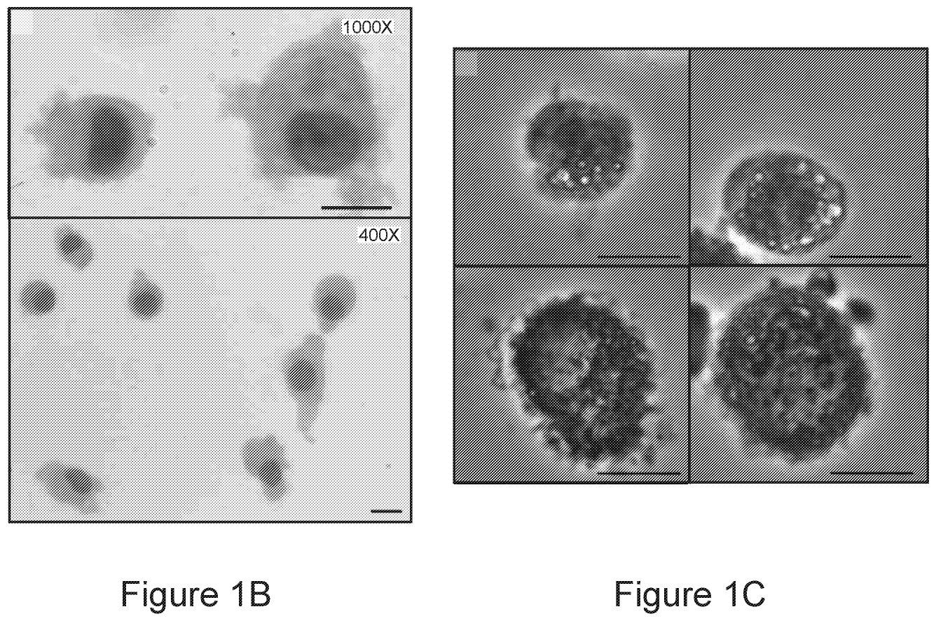

In addition, the labels gran^{high/low} and size^{high/low} may further be determined by microscopic evaluation. For example, size^{high/low} may be determined by microscopy as a difference in mean diameter of at least 1.5, e.g. a 2 or 3 fold difference. For instance, DC-L may have a mean diameter of about 16-30 micron, and DC-S may have a mean diameter of about 5-15 micron. As exemplified herein (see Example 1 and FIG. 1C) iDC-L were determined to have a mean diameter of 20-25 micron, and iDC-S were determined to have a mean diameter of 10-12 micron, as determined by light microscopy.

In another embodiment, there is provided a cell preparation of a substantially pure human monocyte-derived dendritic cell (mdDC) population, selected from the group consisting of:
a) DC-Large (DC-L), characterized, based on their mean size, granularity and high, membrane complexity, respectively, as size^{high}, gran^{high}, complexity^{high}; and
b) DC-Small (DC-S), characterized based on their mean size, granularity and membrane complexity, respectively as size^{low}, gran^{low}, complexity^{low}.

In another embodiment the human mdDC population is a population of immature mdDC or wherein the human mdDC population is a population of mature mdDC.

In certain particular embodiments, the size of the cells is determined by flow cytometry. In certain particular embodiments, the granularity of the cells is determined by flow cytometry. In certain particular embodiments, the membrane complexity of the cells is determined by light microscopy.

“DC maturation” refers to the differentiation of DCs from an immature phenotype to a mature phenotype and is associated with a wide variety of cellular changes, including (1) decreased antigen-capture activity, (2) increased expression of surface MHC class II molecules and costimulatory molecules, (3) acquisition of chemokine receptors (e.g., CCR7), which guide their migration, and (4) the ability to secrete different cytokines (e.g., interleukin-12 [IL-12]) that control T cell differentiation.

Accordingly, the term “immature DC”, as used herein, refers to a dendritic cell having an antigen-presenting ability that is substantially lower, e.g. lower than ½ or lower than ¼ of that of dendritic cells which maturation had been induced by adding LPS (1 μg/mL) and culturing for two days. Furthermore, the immature DC preferably have phagocytic ability for antigens, and more preferably show low (for example, significantly low as compared to mature DCs induced by LPS as described above) or negative expression of receptors that induce the co-stimulation for T cell activation as described herein. Immature DC express surface markers that can be used to identify such cells by flow cytometry or immuno-histochemical staining. Specifically, the characteristics of immature DC-S and DC-L populations of the invention, including surface marker expression, are further described herein.

The term “mature DC”, as used herein, is a cell that has significantly strong antigen-presenting ability for T cell or the like as compared with a dendritic cell in the immature state. Specifically, the mature dendritic cells may have an antigen-presenting ability that is half or stronger, preferably equivalent to or stronger than the antigen-presenting ability of DC in which maturation has been induced by adding LPS (1 μg/mL) and culturing for two days. Mature DC display up-regulated expression of co-stimulatory cell surface molecules and secrete various cytokines. Specifically, mature DCs express higher levels of HLA class I and class II antigens (HLA-A, B, C, HLA-DR) and are generally positive for the expression of CD80, CD83 and CD86 surface markers. The characteristics of mature DC-S and DC-L populations of the invention, including surface marker expression, are further described herein.

In another embodiment, the invention is directed to methods for generating at least one cell preparation of a substantially purified mdDC sub-population selected from the group consisting of iDC-S, mDC-S, iDC-L and mDC-L. In another embodiment, the invention provides methods for generating preparations of substantially purified DC-S (e.g. iDC-S or mDC-S). In another embodiment, the invention provides methods for generating preparations of substantially purified DC-L (e.g. iDC-L or mDC-L). According to some embodiments, the method comprises a) providing a population of human mdDC, and b) isolating the least one mdDC sub-population using cell sorting.

“Cell sorting”, as used herein, encompasses typically immunological-based methods of positive and negative selection, which result in the physical isolation of a cell type, such as a mdDC subset, having a specific cell surface marker or combination of markers using an antibody or an antibody fragment, or a combination of antibodies or antibody fragments, which specifically recognize(s) the marker(s). Examples include, but are not limited to cell sorting by fluorescence-activated cell sorting (FACS), magnetic beads [Magnetic-activated cell sorting (MACS)], columns-based cell sorting, and immuno-panning.

In another embodiment, providing a population of human mdDC is performed by ex-vivo differentiation of monocytes. In another embodiment providing a population of human mdDC is performed by ex-vivo differentiation of monocytes in the presence of GM-CSF and IL-4. Typically, the differentiation is performed in the presence of plasma or serum supplementation, or in serum-free media compatible with DC differentiation. In certain exemplary embodiments, the differentiation is performed in the presence of in the presence of 0.2 to 5% plasma, 200 to 5000 U/mL GM-CSF, and 100 to 2500 U/mL IL-4. In other exemplary embodiments, the differentiation is in the presence of in the presence of 1% plasma, 1000 U/mL GM-CSF, and 500 U/mL IL-4. In certain embodiments, the plasma is autologous plasma. In certain embodiments, the plasma is substituted with an effective amount of serum, or with an effective amount of serum-free cell culture medium supplemented with relevant agents to support cell growth and/or differentiation, as known in the field.

For example, without limitation, human mdDC may be obtained from peripheral blood mononuclear cells, by the following exemplary procedure. PBMC may be enriched by Ficoll gradient separation, and plated in medium containing e.g. 1% autologous plasma onto tissue culture flasks to select for monocytes, which adhere to the plastic surface after a one hour incubation step. Lymphocytes are washed off the flasks, and the monocytes (adherent CD14⁺ cells) are isolated. Alternatively, CD14+ cells may be purified by positive selection for CD14 expression (e.g. using magnetic beads or other forms of cell sorting). The resulting CD14⁺ monocytes are then cultured for several days in the presence of granulocyte-macrophage colony-stimulating factor (GM-CSF) (with or without interleukin (IL)-4). During this period, the monocytes differentiate into immature DCs. On Day 5, the immature DCs are harvested, washed, and isolated. DCs may be stimulated to mature by incubating with maturation signals, e.g. a 1 μg/ml LPS for 24 hours, as described in further detail below. Typically, for the generation of cell vaccines, maturation is induced concomitantly with, or immediately following (e.g. 1 to several hours later), antigen loading, as further detailed below.

In another embodiment, the population of human mdDC is a population of immature mdDC. In another embodiment the least one mdDC sub-population is isolated from immature mdDC in the absence of activation or maturation stimuli (without the addition of such exogenous stimuli under conditions adequate for maturation, e.g. at amounts sufficient to induce DC maturation and/or activation). In another embodiment the least one mdDC sub-population is selected from the group consisting of iDC-S and iDC-L, and is isolated from immature mdDC in the absence of activation or maturation stimuli. According to other embodiments, the cells may be enriched for mature mdDC populations, by incubation in the presence of activation or maturation stimuli (under conditions adequate for maturation).

The expression “conditions adequate for maturation”, as used herein, refers to culturing an immature dendritic cell under conditions suitable to achieve the maturation of said cell. Suitable conditions for maturation are well-known by the skilled in the art. Mature dendritic cells can be prepared by contacting the immature dendritic cells with effective amounts or concentration of a dendritic cell maturation agent.

The terms “dendritic cell maturation agent”, and “maturation agent” as used herein, refer to a compound capable of producing the maturation of the dendritic cell when the dendritic cell is incubated with said compound under conditions adequate for maturation. Dendritic cell maturation agents can include, for example, lipopolysaccharide (LPS), zymosan, a mixture of PgE2, tumor necrosis factor α (TNF-α), and interleukin 1 β (IL-1β), transforming growth factor β (TGF-β), BCG, IFN-γ, monophosphoryl lipid A (MPL), eritoran (CAS number 185955-34-4), TNF-α and their analogs, and combinations thereof. A maturation stimulus includes a maturation agent used under conditions adequate for maturation.

LPS is a ligand for Toll-like receptor (TLR)-4, which is expressed on mammalian DCs, including human DCs. Activation of signal transduction pathways by signaling through TLRs such as TLR4 leads to the induction of various genes including inflammatory cytokines, chemokines, major histocompatibility complex, and upregulation of costimulatory molecules on DCs (i.e., leads to DC maturation). In certain embodiments, DCs are matured in the presence of 1 μg/ml LPS. However, it is to be appreciated that other concentrations of LPS may also be used to achieve comparable results (e.g., maturation of DCs, as determined, e.g., by the expression of CD83 or other maturation marker(s)). Such LPS concentrations include, without limitation, 0.001 μg/ml, 0.005 μg/ml, 0.01 μg/ml, 0.05 μg/ml, 0.1 μg/ml, 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 10 μg/ml, 15 μg/ml, 20 μg/ml, etc. In addition, TLRs have been shown to recognize the bacterial products lipoteichoic acid, peptidoglycan, lipoprotein, CpG-DNA, and flagellin, as well as the viral product double stranded RNA, and the yeast product zymosan, as well as other agents that trigger Toll-like receptors, both extracellular such as TLR4 and TLR2, and/or intracellular such as TLR3, TLR7, and TLR 9.

In other embodiments, other maturation stimuli that do not induce TLR activation, are typically used in combination, e.g. in the form of cytokine cocktails. For example, embodiments of the invention employ the use of a combination of PgE2, TNF-α, IL-1β, or TGF-β.

As exemplified herein, maturation may be achieved by incubation with 2-50 ng/mL LPS, 1-25 μg/mL zymosan, a CKC consisting of 0.2-5 μg/mL PgE₂, 2-50 ng/mL TNF-α and 10-250 ng/mL IL-1β, or 5-125 ng/mL TGF-β. In certain exemplary embodiments, maturation may be achieved by incubation with 10 ng/mL LPS, 5 μg/mL zymosan, a CKC consisting of 1 μg/mL PgE₂, 10 ng/mL TNF-α and 50 ng/mL IL-1β, or 25 ng/mL TGF-β. Each possibility represents a separate embodiment of the invention.

Cell Vaccines and Immuno-Modulating Cell Compositions

In other embodiments, the invention relates to cell vaccines, useful for the treatment or amelioration of cancer or infective diseases, and/or for the induction of immunogenic reactions towards antigens implicated in the etiology and/or pathology of cancer or infective diseases. In some embodiments, the vaccines are DC vaccines, comprising substantially pure mdDC sub-populations as described herein. In other embodiments, the vaccines are T cell vaccines or adoptive T cell therapies, prepared using substantially pure mdDC sub-populations as described herein.

Dendritic cell vaccination is a form of immunotherapy designed to induce T cell-dependent immunity, such as cancer-specific T cell-dependent anti-tumor immunity, that can result in durable complete responses using DCs. A critical step in DC vaccination is the efficient presentation of disease-specific antigens to T cells. DCs are an essential component of vaccination through their capacity to capture, process, and present antigens to T cells. Activated (mature), antigen-loaded DCs initiate the differentiation of antigen-specific T cells into effector T cells that display unique functions and cytokine profiles. “DC maturation” further refers to the differentiation of DCs from an immature phenotype to a mature phenotype and is associated with a wide variety of cellular changes, including (1) decreased antigen-capture activity, (2) increased expression of surface MHC class II molecules and costimulatory molecules, (3) acquisition of chemokine receptors (e.g., CCR7), which guide their migration, and (4) the ability to secrete different cytokines (e.g., interleukin-12 [IL-12]) that control T cell differentiation. According to some embodiments, the cells are pulsed or loaded with antigens associated with the etiology and/or pathology of a disease to be treated.

Thus, in some embodiments, the invention relates to DC vaccines comprising an antigen-pulsed human mdDC population of the invention. The DC vaccines of the invention comprise in some embodiments a cell preparation of the invention, pulsed with at least one disease-associated antigen, said vaccine further comprising a pharmaceutically acceptable carrier, excipient and/or adjuvant

As used herein, the term “antigen-loaded” or “antigen pulsed” in the context of loading a DC with an antigen or antigens (e.g., tumor-associated antigens such as tumor cell lysate), means contacting the DC with the antigen(s) under conditions sufficient to allow the DC to take up (e.g., phagocytose) the antigen(s) and/or express the antigen(s) or peptides derived from the antigen(s) in the context of MHC molecules on the DC cell surface.

The expression “conditions sufficient to allow antigen phagocytosis and/or expression”, as used herein, refers to the incubation of the dendritic cell in a suitable medium and for a sufficient time period to allow the capture of the immunogen and the processing and presentation of said immunogen to other cells of the immune system.

The term “antigen”, as used herein, refers to any molecule that, when introduced into the body, induces a specific immune response (i.e. humoral or cellular) by the immune system.

In various embodiments, cancer and infective diseases to be treated or ameliorated by cell vaccines of the invention may include various tumors and infections (e.g. viral) that are manifested by characteristic antigens typically including T cell epitopes. For example, without limitation, the cancer may be melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and other cancers of the intestinal tract, bone malignancies, connective and soft tissue tumors, and skin cancers. In another embodiment the cancer is selected from the group consisting of melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers. In a particular embodiment the cancer is acute lymphoid leukemia. In other embodiments, the infective disease may be associated with various viral, bacterial fungal and parasitic infections. Each possibility represents a separate embodiment of the invention. Exemplary antigens include, but not limited to, various tumor-associated antigens (TAA) and disease-associated antigens known in the art, including, but not limited to, B7H3, CAIX, CD44 v6/v7, CD171, CEA, EGFRvIII, EGP2, EGP40, EphA2, ErbB2(HER2), and viral antigens present in Cytomegalovirus (CMV), Epstein Barr Virus (EBV), Human Immunodeficiency Virus (HIV), and influenza virus.

Advantageously, cell vaccines according to embodiments of the invention comprise a) at least one preparation of substantially purified DC-L, e.g. mature DC-L obtained by ex-vivo incubation of iDC-L in the presence of at least one disease-associated antigen, and appropriate amounts of cytokines and/or other maturation signals (e.g. LPS); and b) a pharmaceutically acceptable carrier, excipient and/or adjuvant.

In other embodiments, the invention relates to cell compositions useful for the treatment or amelioration of an autoimmune or inflammatory disease, and/or for the induction of a tolerogenic immune reaction towards antigens implicated in the etiology and/or pathology of an autoimmune or inflammatory disease. In some embodiments, the compositions are DC compositions, comprising substantially pure mdDC sub-populations as described herein. In other embodiments, the compositions are T cell compositions (e.g. adoptive transfer therapies), prepared using substantially pure mdDC sub-populations as described herein. Such cell compositions are further referred to herein as the tolerogenic compositions of the invention.

Thus, the invention relates in some embodiments an immune-modulating composition comprising: to comprising: a cell preparation of the invention, pulsed with at least one disease-associated antigen implicated in the etiology and/or pathology of an autoimmune or inflammatory disease and/or with necrotic or apoptotic cells, said cell composition further comprising a pharmaceutically acceptable carrier, excipient and/or adjuvant

According to various embodiments, autoimmune and inflammatory diseases to be treated or ameliorated by the tolerogenic compositions of the invention may be T cell mediated diseases including, but not limited to, autoimmune diseases (e.g. multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease (Crohn's and ulcerative colitis) and autoimmune hepatitis). In other embodiments, the diseases may be inflammatory diseases, particularly chronic, non-resolving diseases. According to particular embodiments, the inflammatory diseases may be e.g. asthma (particularly allergic asthma), hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases). In another embodiment the disease may be graft rejection, e.g. allograft rejection and graft-versus-host disease (GVHD).

In some embodiments, tolerogenic cell compositions according to embodiments of the invention comprise a) at least one preparation of substantially purified DC-S, e.g. mature DC-S obtained by ex-vivo incubation of iDC-S in the presence of at least one disease-associated antigen, and appropriate amounts of cytokines and/or other maturation signals (e.g. LPS); and b) a pharmaceutically acceptable carrier, excipient and/or adjuvant. In another embodiment, said antigen is implicated in the etiology or pathology of a T-cell mediated disease. In another embodiment, said antigen may contain, for example, antigens associated with autoimmune diseases, chronic non-resolving inflammatory diseases, or graft rejection, e.g. autoimmune antigens including but not limited to type II bovine or chicken collagen, HCgp39, lyophilized Escherichia coli extract, the 15-mer synthetic peptide dnaJp1, and citrullinated proteins including but not limited to cit-vimentin, cit-fibrinogen, cit-fibrinogen, and cit-collagen type II, or peptides derived from these citrullinated proteins, insulin, proinsulin, GAD65 (glutamic acid decarboxylase), IA-2 (islet antigen 2; tyrosine phosphatase), the ZnT8 transporter, DiaPep277 and other HSP60-derived peptides, myelin peptides including MBP13-32, MBP83-99, MBP111-129, MBP146-170, MOG1-20, MOG35-55, and PLP139-154.

In other embodiments, the cells are incubated with necrotic or apoptotic cells prior to being administered to the subject. Without wishing to be bound by a single theory or mechanism of action, such incubation may induce tolerogenic functions in the cells, and may thus be useful in the treatment and amelioration of autoimmune and inflammatory diseases. Thus, according to some embodiments, tolerogenic compositions according to embodiments of the invention comprise a) at least one preparation of substantially purified DC-L, e.g. mature DC-L obtained by ex-vivo incubation of iDC-L in the presence of necrotic or apoptotic cells, optionally at least one disease-associated antigen, and appropriate amounts of cytokines and/or other maturation signals (e.g. LPS); and b) a pharmaceutically acceptable carrier, excipient and/or adjuvant. In other embodiments, tolerogenic compositions according to embodiments of the invention comprise a) at least one preparation of substantially purified DC-S, e.g. mature DC-S obtained by ex-vivo incubation of iDC-S in the presence of necrotic or apoptotic cells, optionally at least one disease-associated antigen, and appropriate amounts of cytokines and/or other maturation signals (e.g. LPS); and b) a pharmaceutically acceptable carrier, excipient and/or adjuvant.

In another embodiment the cell composition is a T cell composition, typically an adoptive T-cell composition comprising antigen-specific T-cells.

As used herein, the term “antigen-specific T-cells” refers to T-cells that proliferate upon exposure to the antigen-loaded DC of the present invention, as well as to develop the ability to attack cells having the specific antigen on their surfaces. Such T-cells, e.g., cytotoxic T-cells, lyse target cells by a number of methods, e.g., releasing toxic enzymes such as granzymes and perforin onto the surface of the target cells or by affecting the entrance of these lytic enzymes into the target cell interior. Generally, cytotoxic T-cells express CD8 on their cell surface. T-cells that express the antigen CD4, commonly known as “helper” T-cells, can also help promote specific cytotoxic activity and may also be activated by the antigen-loaded DC of the present invention.

Adoptive T cell therapies according to the invention include T-cell therapies in which T-cells are expanded ex-vivo in the presence of a DC preparation of the invention (e.g. antigen-loaded and/or incubated with apoptotic or necrotic cells) and returned to the patient in large numbers intravenously in an activated state. In some embodiments, the T cells may be T helper cells (CD4⁺) or CTL (CD8⁺). The expanded T-cells that are specific for the antigen presented by the pulsed DC may then be isolated and optionally further expanded and/or stimulated ex-vivo by suitable cytokines (e.g. IL-2) before administration to the patient. In some embodiments, the T cells are histocompatible with the DC. However, in other embodiments (for example when CAR-derived cells are used), the cells may be non-compatible with the subject respect to their MHC-II expression.

The cell populations and compositions and can be formulated for administration in any convenient way for use in treatment of humans. For in vivo administration to humans, the cells and compositions disclosed herein can be formulated according to known methods used to prepare pharmaceutically useful compositions. The DCs can be combined in admixture, either as the sole active material or with other known active materials, (e.g., one or more chemotherapeutic agents) with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. In some embodiments, the cells are formulated for administration by a parenteral route. The term “parenteral” includes subcutaneous injections, intravenous, intramuscular, intra-cisternal injection, or infusion techniques. Also included are intra-tumoral injection, and direct intra-organ injection (e.g., intra-splenic or intra-hepatic injection). For injection or infusion techniques, the DCs may be suspended in any suitable injection buffer, such as, but not limited to, PBS or PBS containing anti-coagulants.

The effective amounts of cells, compositions including pharmaceutical formulations of the present invention include doses that partially or completely achieve the desired therapeutic, prophylactic, and/or biological effect. In a specific embodiment, an effective amount of dendritic cells administered to a patient having a tumor is effective for reducing the size or inhibiting the growth of the tumor in the patient. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

The cell populations and compositions described herein will typically contain an effective amount of DCs, alone, or in combination with an effective amount of any other active material, e.g., a chemotherapeutic agent. Effective amounts, or dosages, and desired concentrations of DCs contained in the compositions may vary depending upon many factors, including the intended use, patient's body weight and age, and route of administration.

Genetically Modified Cells

In other embodiments, the cells in the compositions of the invention may be genetically modified, e.g. to express various targetors (e.g. to a cell, tumor or tissue of interest), co-stimulatory molecules and/or antigens. For example, dendritic cell therapy and other immunotherapies can promote and/or benefit from co-stimulatory molecules which act to provide a stimulatory signal to a T cell to activate T-cell dependent immune responses. During the activation of lymphocytes, co-stimulation is often crucial to the development of an effective immune response. Co-stimulation is required in addition to the antigen-specific signal from their antigen receptors. Non-limiting examples of co-stimulatory molecules include CD80, CD83, CD86, MHC Class II (also referred to in humans as HLA, such as HLA-DR), members of the B7-family of co-stimulatory molecules, CD40, CD40 ligand, CD30, CD30 ligand, 4-IBB receptor, 4-IBB ligand, CD27, FAS receptor, FAS ligand, TRAIL receptor, and TRAIL ligand. In some embodiments of the various aspects described herein, the one or more co-stimulatory molecules is selected from CD80, CD83, CD86, and MHC Class II or HLA-DR. The measurement or detection of co-stimulatory molecules can be performed using methods known in the art.

In other embodiments, cells used in the compositions of the invention may be genetically modified to express chimeric antigen receptors (CARs). A CAR combines the binding site of a molecule that attaches strongly to the antigen being targeted (i.e., a “binding portion”) with the cytoplasmic domains of conventional immune receptors responsible for initiating signal transduction that leads to lymphocyte activation (the “signaling portion”). Most commonly, the binding portion used is derived from the structure of the Fab (antigen binding) fragment of a monoclonal antibody (mAb) that has high affinity for the antigen being targeted. Because the Fab is the product of two genes, the corresponding sequences are usually combined via a short linker fragment that allows the heavy-chain to fold over the light-chain derived peptides into their native configuration, creating a single-chain fragment variable (scFv) region. As many known as the original CARs systems attached an antibody fragment to a T cell, they were also called “T-bodies”. Other possible antigen binding moieties include signaling portions of hormone or cytokine molecules, the extracellular domains of membrane receptors and peptides derived from screening of libraries (e.g. phage display). Suitable antigenic targets for CAR used in the compositions of the invention are disease specific antigens as disclosed herein.

In certain embodiments, the CAR-DC of the present invention comprise “first generation” CAR, having the intracellular domain from the CD3 ζ-chain, which is the primary transmitter of signals from endogenous TCRs. In certain embodiments, the CAR-DC of the present invention comprise “second generation” CAR, further comprising intracellular signaling domain(s) from various co-stimulatory protein receptor(s) in the cytoplasmic tail to provide additional signals to the cell. In certain embodiments, the CAR-DC of the present invention comprise “third generation” CAR, combining multiple signaling domains to augment potency.

The term “antibody” is meant to include both intact molecules as well as fragments thereof that include the antigen-binding site. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

With respect to the cytoplasmic domain, the CAR can be designed to comprise signaling domains of co-stimulatory molecules, e.g. the CD80 and/or CD86 and/or CD40 and/or CD83 signaling domain by itself or combined with any other desired cytoplasmic domain(s) useful in the context of the CAR. The CAR-DC cells of the invention are able to replicate in vivo resulting in long-term persistence that can lead to sustained tumor control. In one exemplary embodiment, the CAR-DC cells of the invention can be generated by introducing a viral vector such as a lentiviral vector comprising a desired CAR, for example a CAR comprising anti-CD19 binding domain, a transmembrane domain, and a cytoplasmic signaling domain, into the cells. Alternatively, a vector is used that is stably maintained in the T cell, without being integrated in its genome. In another embodiment, the CAR-DC cells of the invention can be generated by transduction or transfection of a gene encoding such a CAR molecule in the cell. In certain exemplary embodiments, the CAR comprises scFv of an anti-CD19 antibody linked to 4-1BB (CD137) and CD3ζ signaling domains. In other exemplary embodiments, the CAR may comprise a scFv of anti-CD19 antibody linked to CD28 and CD3ζ signaling domains.

The present invention relates in some embodiments to genetic engineering of dendritic cells with chimeric antigen receptor (or humanized) typically of 2^{nd} generation but also of advanced generations (humanized, multiple costimulatory intracellular, cytokine added (e.g. IL-12 and others) and anti-inhibitory molecules and more.

The present invention relates to both in-vitro interaction of CAR-engineered DCs with tumor samples and further injection into the diseased person, and/or in-vivo enrichment of CAR-engineered DCs populations by growth factors and other material, and in-vivo injection of CAR-engineered DCs not previously exposed to tumor (but carrying the CAR specific to tumor). In-vivo includes I.V., intra-dermal subcutaneous, intra-nodal, intra-tumor, and intra-ventricular (head tumors), and into the CSF.

According to some embodiments, DC populations according to the invention may thereby be engineered to both kill the tumor and digest it for presentation in order to further process additional antigens and present them to T cells. This may enable in some embodiments preventing tumor relapse and providing effective CAR treatment in tumors where hitherto considered to be less amenable for CAR treatment (i.e. lymphoma, CLL, solid tumor).

In certain embodiments, CAR-DC are targeted to one or more cancer-associated antigens by comprising one or more different types of CAR molecules, specifically directed to the relevant cancer-associated antigens. In certain embodiments, the CAR-DC cells of the present invention comprise a CAR specifically directed to CD19. In certain embodiments, the CAR-DC cells of the present invention comprise a CAR specifically directed to CD22. In certain embodiments, the CAR-DC cells of the present invention comprise a CAR specifically directed to CD19 and a CAR specifically directed to CD22. In certain embodiments, the CAR-DC cells of the present invention comprise a CAR specifically directed to CD19 and a different CAR specifically directed to CD22. In certain embodiments, the CAR-DC cells of the present invention comprise a dual-specific CAR directed to CD19 and CD22. In certain embodiments, the CAR-DC cells of the present invention target CD19 and/or CD22 presented by acute lymphoid leukemia (ALL) cells. In certain embodiments, the CAR molecules are chimeric, comprising human-derived and non-human-derived sequences. In certain embodiments, the CAR molecules are humanized, substantially comprising human-derived sequences. In certain embodiments, the CAR molecules are human, consisting of human-derived sequences. Adoptive T-cell therapies include T-cell therapies in which T-cells are expanded in vitro (e.g. using cell culture methods relying on the immunomodulatory action of interleukin-2) and returning these to the patient in large numbers intravenously in an activated state. Adoptive T-cell therapies can also involve genetically engineering a subject's or patient's own T cells to produce recombinant receptors on their surface (CARs).

The preparation of expression constructs or vectors used for delivering and expressing a desired gene product are known in the art. An isolated nucleic acid sequence can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis (see e.g. Sambrook et al., 2001, Molecular Cloning: A Laboratory Manual, Cold Springs Harbor Laboratory, New York; Ausubel, et al., 1989, Chapters 2 and 4).

The construct may also comprise other regulatory sequences or selectable markers, as known in the art. The nucleic acid construct (also referred to herein as an “expression vector”) may include additional sequences that render this vector suitable for replication and integration in prokaryotes, eukaryotes, or optionally both (e.g., shuttle vectors). In addition, a typical cloning vector may also contain transcription and translation initiation sequences, transcription and translation terminators, and a polyadenylation signal.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives. These may serve as vector backbone for the constructs useful in embodiments described herein.

Expression vectors containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Barr virus include pHEBO and p2O5. Other exemplary vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells. These may serve as vector backbone for the constructs of the present invention.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinarily skilled artisan and as such, no general description of selection considerations is provided herein.

Recombinant viral vectors are useful for in vivo expression of the genes of the present invention since they offer advantages such as lateral infection and targeting specificity. Lateral infection is inherent in the life cycle of retrovirus, for example, and is the process by which a single infected cell produces many progeny virions that bud off and infect neighboring cells. The result is the rapid infection of a large area of cells, most of which were not initially infected by the original viral particles. This is in contrast to vertical-type infection in which the infectious agent spreads only through daughter progeny. Viral vectors can also be produced that are unable to spread laterally. This characteristic can be useful if the desired purpose is to introduce a specified gene into only a localized number of targeted cells.

Retroviral-derived vectors include e.g. lentiviral vectors. “Lentiviral vector” and “recombinant lentiviral vector” are derived from the subset of retroviral vectors known as lentiviruses. Lentiviral vectors refer to a nucleic acid construct which carries, and within certain embodiments, is capable of directing the expression of a nucleic acid molecule of interest. The lentiviral vector includes at least one transcriptional promoter/enhancer or locus defining element(s), or other elements which control gene expression by other means such as alternate splicing, nuclear RNA export, post-translational modification of messenger, or post-transcriptional modification of protein. Such vector constructs must also include a packaging signal, long terminal repeats (LTRS) or portion thereof, and positive and negative strand primer binding sites appropriate to the lentiviral vector used (if these are not already present in the retroviral vector). Optionally, the recombinant lentiviral vector may also include a signal which directs polyadenylation, selectable markers such as Neo, TK, hygromycin, phleomycin, histidinol, or DHFR, as well as one or more restriction sites and a translation termination sequence. By way of example, such vectors typically include a 5′ LTR, a tRNA binding site, a packaging signal, an origin of second strand DNA synthesis, and a 3′LTR or a portion thereof.

“Lentiviral vector particle” may be utilized within the present invention and refers to a lentivirus which carries at least one gene of interest. The retrovirus may also contain a selectable marker. The recombinant lentivirus is capable of reverse transcribing its genetic material (RNA) into DNA and incorporating this genetic material into a host cell's DNA upon infection. Lentiviral vector particles may have a lentiviral envelope, a non-lentiviral envelope (e.g., an amphotropic or VSV-G envelope), or a chimeric envelope.

It should be understood that according to the principles of the present invention, any non-destructive method known in the field to insert genetic material, specifically DNA, to living cells, specifically dendritic cells, is considered to be applicable to deliver the CAR gene into the target dendritic cells. For example, transfection, transduction, infection and electrophoresis are considered relevant.

Therapeutic Use

In another embodiment, the invention relates to a cell vaccine of the invention, for use in a method for the treatment or amelioration of cancer or an infective disease in a subject in need thereof. In another embodiment said disease-associated antigen is a tumor-associated antigen, for use in a method of treating cancer in said subject. In another embodiment the invention relates to a cell vaccine of the invention, for use in a method for inducing or enhancing an immunogenic reaction towards antigens implicated in the etiology and/or pathology of cancer or an infective disease in a subject in need thereof. In another embodiment said antigen is a tumor-associated antigen. In various embodiments, said tumor is selected from the group consisting of melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers, wherein each possibility represents a separate embodiment of the invention.

In another embodiment, the invention relates to an immuno-modulating cell composition of the invention, for use in a method for the treatment or amelioration of an autoimmune or inflammatory disease in a subject in need thereof. In another embodiment the invention relates to an immuno-modulating cell composition of the invention, for use in a method for induction of a tolerogenic immune reaction towards antigens implicated in the etiology and/or pathology of an autoimmune or inflammatory disease in a subject in need thereof. In another embodiment said antigen is implicated in the etiology or pathology of a T cell mediated disease selected from the group consisting of: autoimmune diseases, chronic non-resolving inflammatory diseases, and graft rejection. In other embodiments, said autoimmune disease is selected from the group consisting of multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease and autoimmune hepatitis, wherein Each possibility represents a separate embodiment of the invention.

It should be understood that the cell vaccines and immuno-modulating cell compositions according to the present invention may include mature DCs according to the present invention, immature DCs according to the present invention, and any combination thereof, as determined to be beneficial on a case-to-case basis.

It should be understood that the T cell preparation activated in the presence of the cell preparation according to the present invention may be activated by mature DCs according to the present invention, immature DCs according to the present invention, and any combination thereof.

As used herein, the term “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; and/or (2) inhibiting the state, disorder or condition, i.e., arresting, reducing or delaying the development of the disease or a relapse thereof or at least one clinical or sub-clinical symptom thereof; and/or (3) relieving the disease, i.e., causing regression of the state, disorder or condition or at least one of its clinical or sub-clinical symptoms.

In another embodiment, the invention provides a method for the treatment or amelioration of cancer or an infective disease in a subject in need thereof, comprising administering to the subject an effective amount of a cell vaccine of the invention.

In another embodiment, the invention provides a method for inducing or enhancing an immunogenic reaction towards antigens implicated in the etiology and/or pathology of cancer or an infective disease in a subject in need thereof, comprising administering to the subject an effective amount of a cell vaccine of the invention.

In another embodiment, the invention provides a method for the treatment or amelioration of an autoimmune or inflammatory disease in a subject in need thereof, comprising administering to the subject an effective amount of a tolerogenic composition of the invention.

In another embodiment, the invention provides a method for induction of a tolerogenic immune reaction towards antigens implicated in the etiology and/or pathology of an autoimmune or inflammatory disease in a subject in need thereof, comprising administering to the subject an effective amount of a tolerogenic composition of the invention.

Typically, cell preparations to be used in cell vaccines and tolerogenic compositions of the invention are substantially viable. Viable DC are preferred in some embodiments as they retain the ability to migrate to a disease site or tissue. However, in some embodiments the use of cells that have initiated an apoptosis process is contemplated, e.g. in cell vaccines comprising DC-L preparations.

In another embodiment, the cells to be administered to the subject in the methods of the invention are autologous. In another embodiment, the cells to be administered to the subject in the methods of the invention are allogeneic. According to certain embodiments of the methods of the invention, the cell composition is histocompatible with the subject (e.g. autologous cells or MHC II-matched allogeneic cells). According to other certain embodiments of the methods of the invention (e.g. when using CAR-derived cells), the cell composition is not histocompatible with the subject.

In another embodiment there is provided a kit for the preparation of a cell vaccine or tolerogenic composition, comprising isolated cell populations and/or means for their preparation as described herein.

The dosage of the cell compositions and formulations disclosed herein may vary, depending upon the nature of the disease, the patient's medical history, the frequency of administration, the manner of administration, the clearance of the cells from the host, and the like. The initial dose may be larger, followed by smaller maintenance doses. The dose may be administered as infrequently as weekly or biweekly, or fractionated into smaller doses and administered daily, semi-weekly, bi-weekly, quarterly, etc., to maintain an effective dosage level. Preliminary doses can be determined according to animal tests, and the scaling of dosages for human administration can be performed according to art-accepted practices. In certain embodiments, a subject may be administered 1 dose, 2 doses, 3 doses, 4 doses, 5 doses, 6 doses or more of a DC-based composition described herein.

Typical dosages (effective amounts) of DCs for administration to a patient may range from 1*10³ to 1*10⁸ cells per dose, although more or less cells may be used. In certain embodiments, the number of dendritic cells ranges from 1*10⁴ to 1*10⁸, in certain embodiments from 1*10⁵ to 1*10⁸, still in certain embodiments from 1*10⁶ to 1*10⁸, and in certain embodiments from 1*10⁶ to 1*10⁷. However, other ranges are possible, depending on the patient's response to the treatment. Moreover, an initial dose may be the same as, or lower or higher than subsequently administered doses of the DCs.

A variety of means for administering cells to subjects are known to those of skill in the art. Such methods can include systemic injection, for example i.v. injection, or implantation of cells into a target site in a subject. Cells can be inserted into a delivery device which facilitates introduction by injection or implantation into the subject. Such delivery devices can include tubes, e.g., catheters, for injecting cells and fluids into the body of a recipient subject. In some embodiments, the tubes additionally have a needle, e.g., through which the cells can be introduced into the subject at a desired location. The cells can be prepared for delivery in a variety of different forms. For example, the cells can be suspended in a solution or gel or embedded in a support matrix when contained in such a delivery device. Cells can be mixed with a pharmaceutically acceptable carrier or diluent in which the cells remain viable.

Pharmaceutically acceptable carriers and diluents include saline, aqueous buffer solutions, solvents and/or dispersion media. The use of such carriers and diluents is known in the art. The solution is preferably sterile and fluid. Preferably, prior to the introduction of cells as described herein, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Direct injection techniques for cell administration can also be used to stimulate transmigration of cells through the entire vasculature, or to the vasculature of a particular organ, such as for example liver, or kidney or any other organ. This includes non-specific targeting of the vasculature. One can target any organ by selecting a specific injection site, e.g., a liver portal vein. Alternatively, the injection can be performed systemically into any vein in the body. If so desired, a mammal or subject can be pre-treated with an agent, for example an agent administered to enhance cell targeting to a tissue (e.g., a homing factor) can be placed at that site to encourage cells to target the desired tissue. For example, direct injection of homing factors into a tissue can be performed prior to systemic delivery of cells.

In another embodiment, the invention is directed to methods for distinguishing between cell sub-populations, comprising: a) providing a cell preparation b) subjecting the preparation to at least one apoptotic stimulus and c) identifying distinct cell sub-populations in the preparation based on distinct changes in their morphology, phenotype and/or functions following the at least one apoptotic stimulus.

The following examples are presented in order to more fully illustrate some embodiments of the invention. They should, in no way be construed, however, as limiting the broad scope of the invention.

### EXAMPLES

Materials and Methods

Media & Reagents

Cell culture medium consisted of RPMI 1640 (Invitrogen-Gibco, Carlsbad, Calif., USA) supplemented with 1% L-glutamine and 1% penicillin/streptomycin (Biological Industries, Kibbutz Beit-Haemek, Israel). Fluorescent Annexin V was obtained from MBL Inc. (Woburn, Mass., USA). Sytox blue (cat S11348), DiD (cat D307), fluorescent dextran (MW 10,000, cat D22910), soluble Alexa Fluor 488 hydrazide (cat A10436), DQ-ovalbumin (cat D82053), fluorescent E. coli (cat E13231), fluorescent zymosan (cat Z23373), and CFDA-SE (“CFSE”, cat C1157) were obtained from Invitrogen-Molecular Probes. Fluorescent latex beads (cat L5405), DiOC₆(3) (cat 318426), carbonyl cyanide 3-chlorophenylhydrazone (“CCCP”, cat C2759), PgE₂ (cat P0409), zymosan (cat Z4250), hematoxylin (cat GH5116), eosin (cat H40216), crystal violet (cat C0775), and lipopolysaccharide from E. coli (cat L6529) were purchased from Sigma Aldrich (St. Louis, Mo., USA). Propidium iodide was obtained from both Invitrogen-Molecular Probes and Sigma-Aldrich. IL-4, GM-CSF, TNF-α, TGF-β, and IL-1β were purchased from PeproTech Inc. (Rocky Hill, N.J., USA). Primary antibodies were obtained from Dako (Glostrup, Denmark), Becton Dickinson (Franklin Lakes, N.J., USA), BioLegend (San Diego, Calif., USA), and AbD Serotec-MorphoSys (Kidlington, UK). Secondary antibodies were obtained from Jackson ImmunoResearch (West Grove, Pa., USA) and Invitrogen-Molecular Probes. Mouse and goat Ig were from Jackson ImmunoResearch.

Isolation of PMN and Monocytes

The two options for obtaining leukocytes were 1) Buffy coats of healthy donors, or 2) Peripheral blood of healthy volunteers). For the isolation of PMN, RBC were sedimented by adding 6% hetastarch in a 0.9% NaCl solution (Hetasep, Stem Cell Technologies, Vancouver, Canada) and kept at RT for up to 40 min. The leukocyte-rich upper layer of the suspension was then collected and centrifuged on a density gradient using Ficoll (Pharmacia, Uppsala, Sweden). Residual erythrocytes were removed by hypotonic lysis. For the isolation of monocytes, PBMC were prepared using a Ficoll density gradient. Next, positive selection using CD14 magnetic beads was performed according to the manufacturer's instructions (Becton Dickinson). For both PMN and monocytes, purity exceeded 95%, and >95% excluded PI.

Generation of Monocyte-Derived Dendritic Cells

Immature mdDCs were generated from the CD14⁺ selected fraction of PBMC mentioned above. Briefly, monocytes were plated in the central wells of 12-well plates at a concentration of 1.25×10⁶/1.5 mL culture medium, in the presence of 1% autologous plasma, GM-CSF (1000 U/mL), and IL-4 (500 U/mL). Every other day, 0.15 mL was removed from the medium and 0.25 mL medium containing plasma, IL-4, and GM-CSF was added. iDCs were obtained at day 6. To obtain mature DCs, iDCs at day 6 received fresh media and cytokines together with either 10 ng/mL LPS, 5 μg/mL zymosan, or a cytokine cocktail (CKC) consisting of 1 μg/mL PgE₂, 10 ng/mL TNF-α and 50 ng/mL IL-1β. Alternatively, TGF-β was added at 25 ng/mL.

Induction and Detection of Cell Death

Viability assays were performed as previously described. Briefly, staining buffer consisted of 140 mM NaCl, 4 mM KCl, 0.75 mM MgCl₂, and 10 mM HEPES. Annexin V, DiOC₆(3), PI and SB were titrated to obtain optimal signal to noise. Annexin V and calcium were added 10 minutes before analysis of the cells; calcium was added to reach 1.5 mM. DiOC₆(3) was added 30 minutes before cell extraction from culture. DiOC₆(3) was titrated and verified with positive controls using CCCP. To induce apoptosis in PBMCs, they were collected by leukopheresis and frozen. On the day of use they were thawed and exposed to methylprednisolone (Sigma Aldrich), following which they acquired an early apoptotic phenotype (>60% Annexin V⁺, <5% PI⁺). To induce apoptosis in PMN, they were incubated at 4×10⁶ per mL in 1 mL RPMI in 24-well plates for 14±2 hours. For necrotic PMN, the cells were incubated at 56° C. until >80% were trypan blue-positive. For uptake assays, PMN were stained with DiD according to the manufacturer's instructions, followed by cell death induction as described above.

Phagocytosis and Interaction Studies

Phagocytosis targets were added to the DCs for 8-12 hours. The following concentrations were used: soluble Alexa Fluor 488 hydrazide, fluorescent dextran, and DQ-ovalbumin, 1 mg/mL; fluorescent E. coli, 10 particles per DC; fluorescent zymosan, 3 particles per DC; fluorescent latex beads, 15 or 50 beads per DC. To control for preparation of the samples, control targets were incubated on ice for 30 minutes. DiD-labeled apoptotic or necrotic PMN were added at 4 cells per DC after a washing step. After incubation with the labeled cells for 8-12 hours, samples were stained with either HLA-DR or DCSIGN for specific identification of the DCs and then analyzed using flow cytometry. For interaction studies, apoptotic or necrotic cells were washed and added to the DCs at 4 cells per DC, followed by 24-hour incubation before analysis. When indicated, LPS was added 6 hours after adding the apoptotic cells. To phenotype the DCs after their interaction with dying cells, antibody cocktails were designed so that at least one label would be of a highly expressed DC-specific marker, to exclude free apoptotic cells that entered the scatter gates.

Cytometry

FACScan™, FACScalibur™ flow cytometers used, and primarily the LSR II™ (Becton Dickinson), and Imagestream™ 100 (Amnis, Seattle, Wash., USA). Compensation was performed in software. For microscopy, a Nikon Eclipse E400 microscope equipped with a Micropublisher 3.3 RTV CCD color camera (Q Imaging, Surrey, BC, Canada) was used. Cells were prepared by cytocentrifugation and then fixed in 95% ethanol, followed by a standard hematoxylin and eosin (H&E) staining protocol. Alternatively, the cells were imaged live after adding 10% of a 1 mg/mL solution of crystal violet. Several measures were taken in order to ascertain the reliability of the results. 1) antibody competition studies were performed, which confirmed that at the stages of cell death studied, antibody binding is specific. 2) Pulse area vs height doublet discrimination was used to exclude cell pairs that could bias the readings. 3) Isotype controls were extensively used to assess and identify nonspecific binding. Bad samples, identified by abnormally high isotype binding, were excluded. For the remaining samples, contributions of the isotypes (which include auto-fluorescence) were mathematically subtracted from the calculations. 4) Directly conjugated antibodies were preferentially used. 5) Antibody labeling was performed in the presence of 75 μg/mL of mouse Ig. Alternatively, purified antibodies were used, in which case secondary staining was performed using goat anti-mouse antibodies in the presence of 75 μg/mL goat Ig. 6) The staining buffer consisted of PBS without calcium, supplemented with HEPES and 1% fetal calf serum (all from Biological Industries). 7) As shown in FIG. 1A, terminal apoptotic cells and fragments were successfully gated out from the DC clusters.

Cell Sorting

Sorting was performed on a FACSAria I (Becton Dickinson). iDCs were taken at day 6 and stained with CD47, CD11c, and DCSIGN, as well as SB. They were then sorted by creating hierarchical gating that selected DC-S as the cells expressing the lowest levels of all three markers, and DC-L as the cells expressing the highest levels. For monocytes, PBMC were stained with CD14 and CD16, as well as SB. They were then sorted into CD14⁺CD16⁻ and CD14^{dim}CD16⁺ populations, and cultured for 6 days as described above for differentiation into DCs.

RNA & Microarrays

Following sorting, the DCs were replated and incubated for 24 hours in the presence of autologous plasma, GM-CSF, and IL4. LPS was added at 10 ng/mL when maturation was induced. Then RNA was extracted using Quiagen's (Hilden, Germany) RNeasy kit according to manufacturer instructions. RNA from 3 donors was pooled and then processed and analyzed at the microarray facility of the Israeli National Strategic Center for Gene Therapy in the Goldyne Savad Institute of Gene Therapy of our institution, using Human Gene 1.0 ST microarrays (Affymetrix, Santa Clara, Calif., USA). Preprocessing of the microarray data was done using RMA. Probeset intensities were transformed to s logarithmic scale and a cutoff of 4 was used. Probesets were considered to be differentially expressed if they showed a linear fold change ≥2. Probes lacking refseq identities were excluded, and instances of multiple probes corresponding to the same genes were collated together.

Data Analysis

Software analysis of flow cytometry data was performed using FCS Express (De Novo Software, Toronto, Canada), including software compensation. When studying phenotype upon cell death, relevant isotypes were used and measured as the rest of the surface markers, gated as SB- or PI-high, low or negative, as detailed hereinbelow. Then, upon summarizing the data, isotype MFI (which includes auto-fluorescence) was subtracted from marker MFI. Heat-maps were created using “Heat-map Builder” (Stanford University School of Medicine). Statistical analysis was performed with Excel (Microsoft, Seattle, Wash., USA). The Student's two-tailed t-test for statistical analysis with a p value of 0.05 for the significance cutoff was used. When applicable, a paired t-test was used, as indicated in the figure legends.

### Example 1. Forward and Side Scatter Analysis Reveals Two DC Subsets, DC-Small and DC-Large, which are Morphologically Different

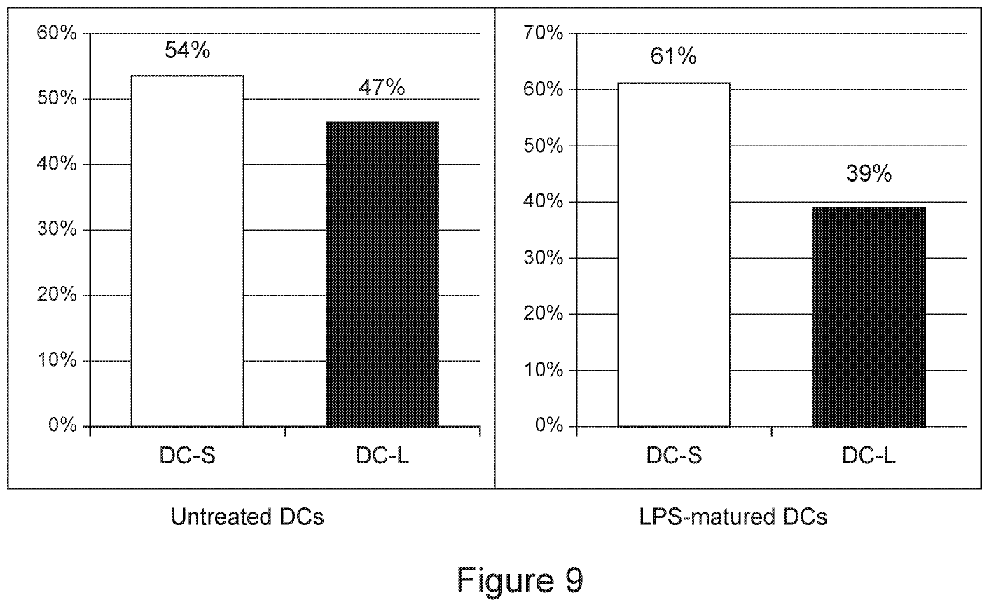

Two clusters of cells were identified on the flow cytometry light scatter plots (FIG. 1A), termed “DC-small” (DC-S) and “DC-large” (DC-L). These two populations appeared in all the flow cytometers used, although they are resolved to varying extents depending on differences in the machines' light-collecting optics. Among immature DCs (iDCs), iDC-S comprise, on average, about 54% of the total cells (FIG. 9), and iDC-L comprise, on average, about 47% of the total cells. After induction of maturation with LPS, the mean percentage of DC-S increases to an average of about 61%, while the mean percentage of DC-L decreases to an average of about 39% (FIG. 9).

Given that cell death is commonly accompanied by changes in light scatter characteristics, it was examined whether these two populations represent different viability states. Overall, less than 5% of DCs were trypan blue-positive on counts, and both DC-S and DC-L were largely viable cells as assayed by Annexin V, propidium iodide (PI), and Sytox Blue (SB). These findings were confirmed using DiOC₆(3), a mitochondrial membrane potential sensitive dye. Thus, cell viability does not account for the DC-S and DC-L differences.

Forward scattering is a useful approximation of cell size. DCs were shown to be significantly heterogenic in size when imaged, supporting the fact that there are smaller and larger cells (FIG. 1B). Upon sorting, morphological differences in DC-S and DC-L are reproduced in the distinct populations (FIG. 1C). DC-L are larger, they show greater membrane complexity, and are more granular. As can be seen in FIG. 1C, iDC-L were determined by microscopic evaluation to have a mean diameter of about 20-25 micron and iDC-S were determined to have a mean diameter of about 10-12 micron.

Since human monocytes are comprised of two main populations, CD16⁺ and CD16⁻, it was examined whether they were responsible for the development of DC-S and DC-L. To that end, peripheral blood monocytes were sorted into CD14⁺CD16⁻ and CD14^{dim}CD16⁺ populations, and differentiated them into DCs using the same protocol. Both monocyte subpopulations gave rise to DC-S and DC-L, implying that the new clusters of cells do not overlap with the “classical” CD16-clustered populations.

### Example 2. DC-S and DC-L Express Different Levels of Surface Markers and Respond Differently to Maturation Stimuli

The expression of surface markers was next characterized in these two populations using an extensive panel of antibodies. When DCs are immature (culture day 6), the relative expression of surface markers shows a broad spectrum of their relative prevalence, ranging from a DC-L/DC-S ratio of 94 to 135 (FIG. 2). Both DC-S and DC-L express CD14 dimly and DCSIGN strongly, indicating that both are fully differentiated DCs. Both DC-S and DC-L express low levels of CCR7, CD83, and CD25, and both upregulate these and other maturation surface markers upon stimulation. This confirms that there are two subpopulations that are initially immature rather than one population of DCs at different maturation stages.

When the DCs were tested following stimulation with a cytokine cocktail (CKC) of PgE₂, TNF-α and IL-1β; LPS; zymosan; or TGF-β, a striking diversity was noted, and in some cases even a divergence of DC-S and DC-L response patterns (FIG. 3, in which different surface markers are presented in FIG. 3A and FIG. 3B).

When looking at all the mature DCs (i.e. before analyzing DC-S and DC-L separately), specific responses to LPS and CKC were seen, which is consistent with the known literature. Yet when analyzing the subpopulations separately, for both CKC and LPS, as seen in FIG. 3B, subset-specific changes were observed. DC-L shows higher expression of stimulatory surface markers CCR7, CXCR4, HLA-ABC, and CD25, or HLA-DR, CD86, and CD54, after CKC or LPS stimulation, respectively.

In contrast, stimulation does not alter the DC-L/DC-S ratio of CD135 or αᵥβ₃ integrin expression seen with iDCs. For CCR5, E-cadherin, or CD206, the DC-L/DC-S ratio for iDCs differs greatly from the ratios of the stimuli used

In other cases, such as DCSIGN or CD11b, the ratio for TGF-β is similar to that seen for iDCs (DC-L>DC-S), whereas LPS, zymosan, and CKC move the DC-L/DC-S ratio of these markers towards 100. There are other cases, for example CD14, where the response is markedly different for a single stimulus, in this case zymosan.

While they are immature, both iDC-S and iDC-L strongly express CD141 (BDCA-3) but low levels of DNGR1 (FIG. 2). The overall level of expression increases by 50% and 100% for CD141 and DNGR1, respectively, after stimulation with LPS, but the DC-L/DC-S ratios remain unchanged at 120 and 95 (FIG. 2).

In summary, upon challenge, DC-S and DC-L show differing responses that may be surface marker- and stimulus-specific. Importantly, these results are consistent despite the fact that the DCs used here were derived from tens of random human donors whose primary cells underwent up to 8 days of culture during their differentiation and stimulation.

### Example 3. RNA Microarrays Reveal a Variety of Genes that are Differentially Expressed on DC-S and DC-L

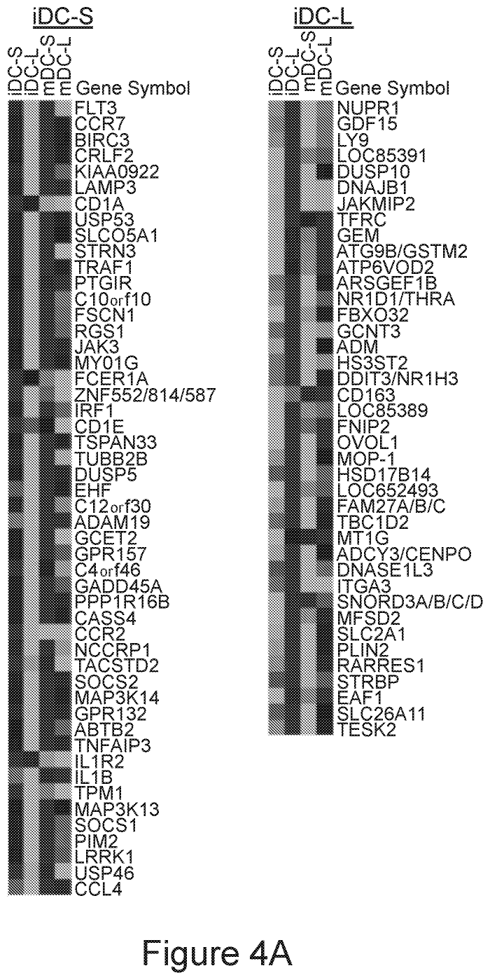

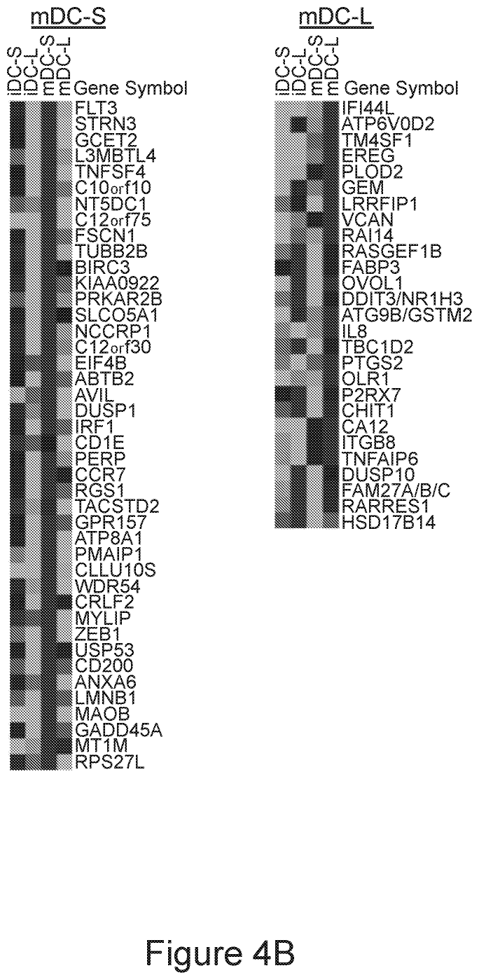

To better understand the nature of the differences between DC-S and DC-L, their transcriptional profiles were analyzed. In FIG. 4, heat-map representations of the four samples studied are shown: DC-S and DC-L at the immature stage (iDC-S and iDC-L, respectively) and at the mature stage (mDC-S and mDC-L, respectively). FIG. 4 shows the absolute expression level of genes of interest, in comparison to all four samples. In the immature DC-S sample, several immunologically relevant gene products were found that are in the bottom third of the immature DC-L/DC-S phenotypic expression scale among iDCs. This provides an important correlation between the transcriptome and the observed phenotype. Several other immunologically relevant genes are also present in the iDC-S sample. The iDC-L sample also had important immune genes, as well as a large number of genes whose function is not yet completely understood.

When comparing DC-S and DC-L at the LPS-matured stage, a repetition of genes was observed, showing the stability of their transcriptomes and providing further evidence of their distinct, stable identities. In the mature DC-S sample many genes were found that are not present at the immature stage. In the mature DC-L sample there are a considerable number of immunologically relevant genes, as well as an abundance of apoptosis-related genes and genes related to the uptake of dead cells.

### Example 4. Surface Marker Expression Upon Cell Death is Different for DC-S and DC-L

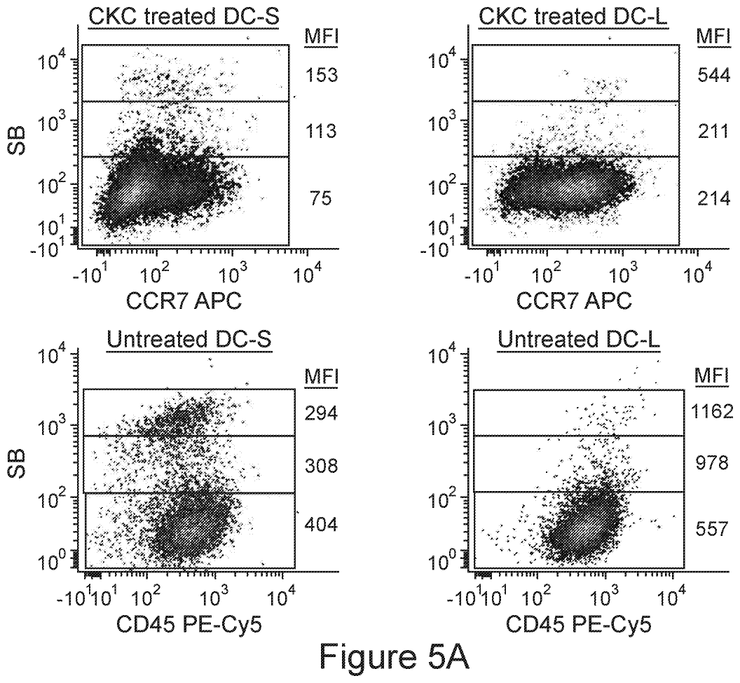

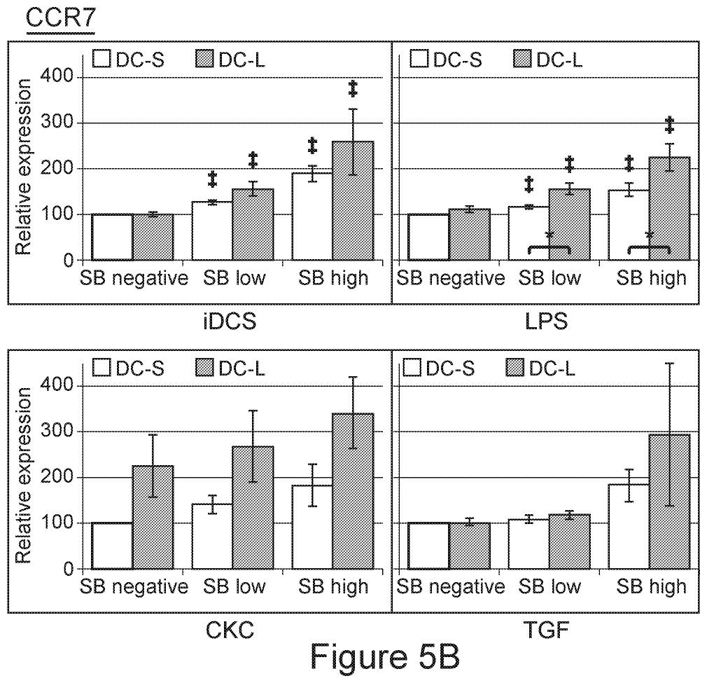

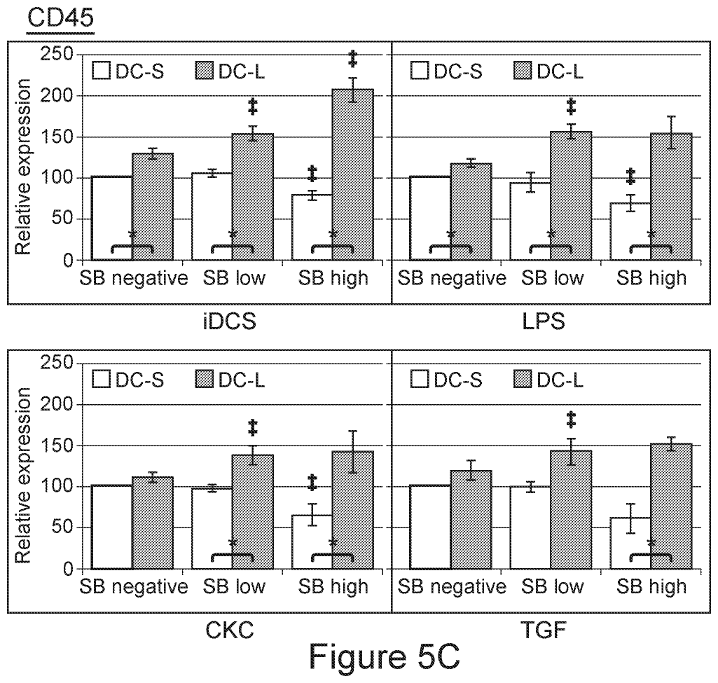

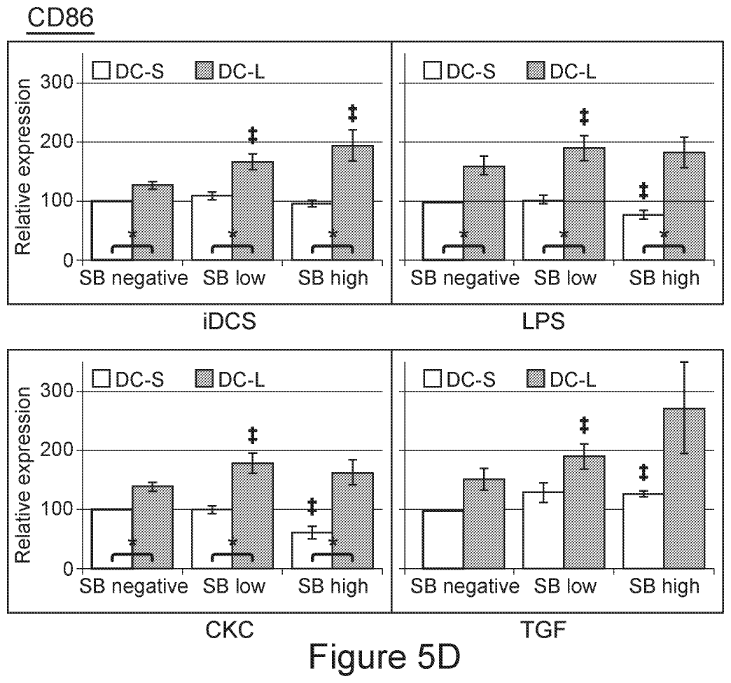

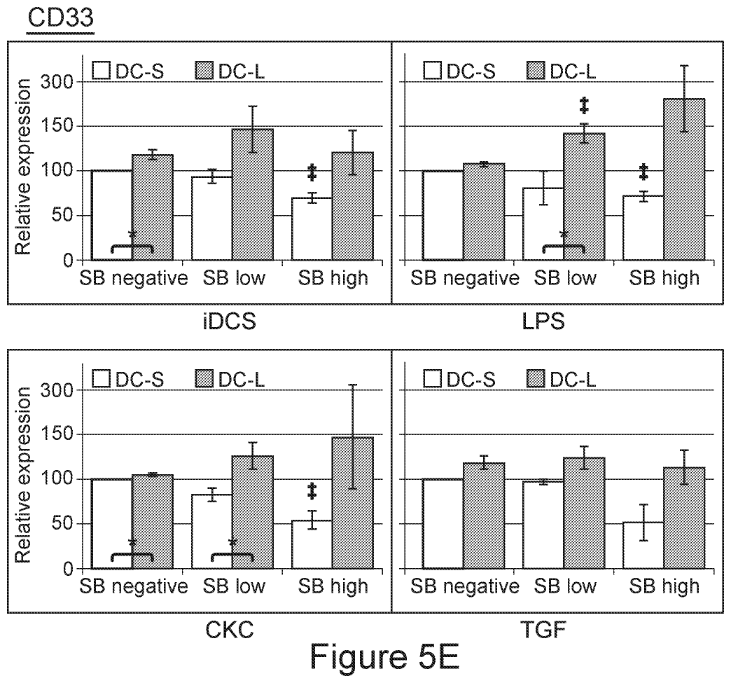

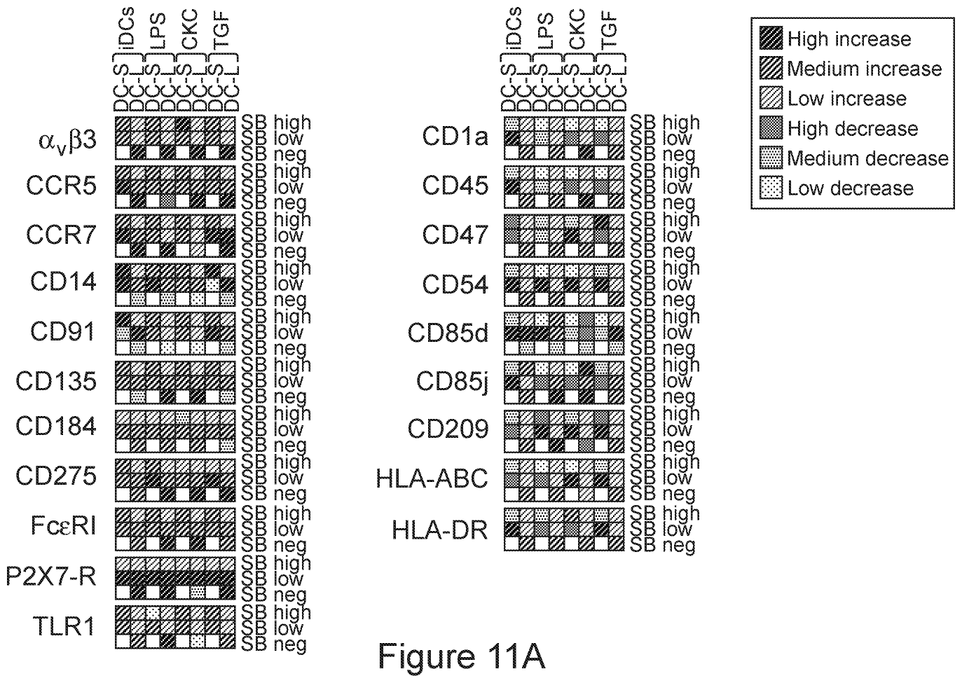

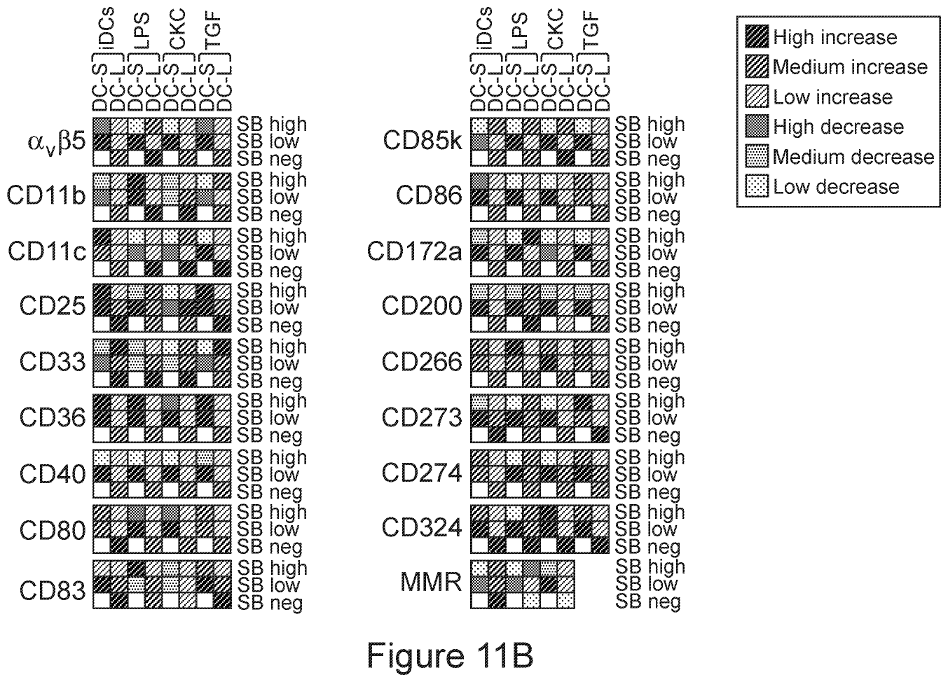

Since viable DC-S and DC-L differ in the level of surface marker expression, it was examined whether there would also be differences upon cell death. To investigate this question, all of the samples were co-stained with propidium iodide (PI), and/or Sytox Blue (SB). It has been shown that PI fluorescence intensity, as well as the intensity of other membrane-excluded, nucleic acid-specific fluorescent dyes, correlates with the advance of cell death. Both SB and PI, were titrated and tested, including PI+SB double staining, with equivalent results for both dyes. The cells were then classified according to their uptake of SB or PI as negative, low, or high, and the median fluorescence intensity (MFI) of the surface marker of interest for each state was measured (FIG. 5). Surprisingly, consistent patterns were observed showing significant differences between DC-S and DC-L phenotypes upon death. As can be seen, when the cells advance in the death process, their level of expression of different markers change. For CCR7, both DC-S and DC-L increase their expression upon advancing cell death. In this case, CKC-treated cells are shown since they have the highest expression of CCR7 allowing for the clearest visualization. In the case of CD45, in contrast, whereas DC-L still increase the expression levels upon advancing cell death, for DC-S it actually decreases. Three general patterns of expression were identified, which are shown in the bar charts in FIG. 5: Pattern 1, surface marker expression increases for both DC-S and DC-L as cell death progresses (FIG. 5B, CCR7); Pattern 2, surface marker expression increases for DC-L while it decreases for DC-S as cell death progresses (FIG. 5C, CD45); and Pattern 3, surface marker expression shows a mixed pattern as cell death progresses with behavior dependent on the stimuli used (FIG. 5D, CD86), or surface marker expression does not change monotonically with advancing cell death (FIG. 5E, CD33). Of note, in most cases the mixed pattern (Pattern 3) was similar to Pattern 2. FIG. 11 shows these results aggregated for all markers tested in a heat-map representation.

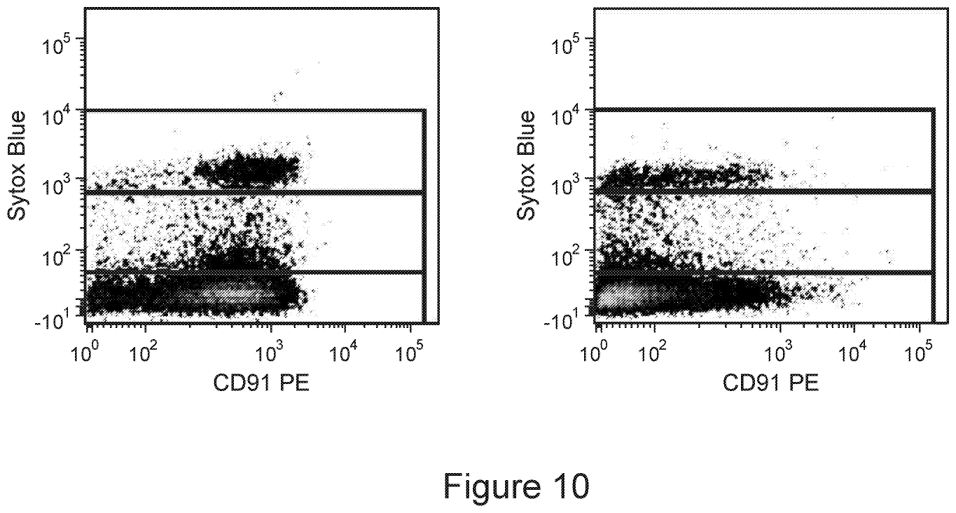

The cells shown represent DCs undergoing spontaneous cell death. It is possible that non-specific antibody binding could affect the results in dying cells. At the stages of programmed cell death (PCD) studied, using a protocol that minimizes non-specific binding (see Materials and Methods), the antibodies do bind specifically (FIG. 10). When cells enter more advanced stages of cell death, their light scatter properties change, and they exit the analysis gates (FIG. 1). PI and SB start entering the cells at an early apoptotic stage, shortly after they become Annexin V positive. They then progressively acquire more PI or SB as they advance in the cell death process. This is the rationale behind the use of PI or SB intensity (in contrast to merely positive vs. negative) as a marker of advancing cell death. Of note, experiments performed staining surface markers together with Annexin V and PI or SB showed that there are only very small differences in the level of surface marker expression when advancing from the Annexin V single positive stage to the PI or SB low stage. Therefore, for the sake of simplicity, further experiments were performed using only PI or SB when studying marker expression changes during cell death.

In summary, the expression of surface markers changes upon the DCs' cell death. This is not a uniform, but a heterogeneous process, with different markers and different stimuli affecting the direction and magnitude of the changes differentially for DC-S and DC-L.

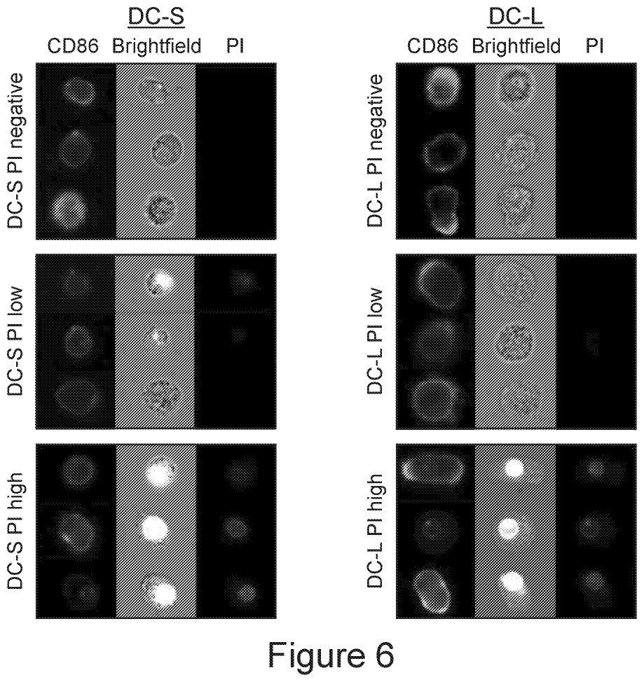

To further assess DCs condition at the SB- or PI-low and SB- or PI-high stages, the cells were stained with CD86 and PI, and analyzed live using an Imagestream™ cytometer (Amnis, EMD Millipore, Seattle, Wash., USA). As can be seen in FIG. 6, morphological differences between DC-S and DC-L in size, shape, and intracellular and membrane complexity were observed. As the cells advance to the PI-low stage, there are no morphological changes to be observed. This was confirmed by a battery of quantitative morphological measurements provided by the Imagestream analysis software. This comes to confirm that the PI-low stage indeed corresponds to an early stage of apoptosis. Only at the PI-high stage are morphological changes observed, such as slight shrinkage of DC-S and loss of membrane and cytoplasmic complexity of DC-L; PI fluoresces strongly in the nucleus, which becomes refractive. Nevertheless, even these “PI-high cells” are whole cells, without blebs and with intact nuclei.

In summary, DC-S and DC-L show differing changes in phenotype upon entering the cell death process. The changing phenotypes are affected by maturation state and stimulus. These changes occur before and at the early phases of acquisition of morphological evidence of cell death.

### Example 5. DC-S and DC-L have Distinct Capabilities for Phagocytosis, but DC-L is Better at Antigen Processing and Uptake of Dying Cells

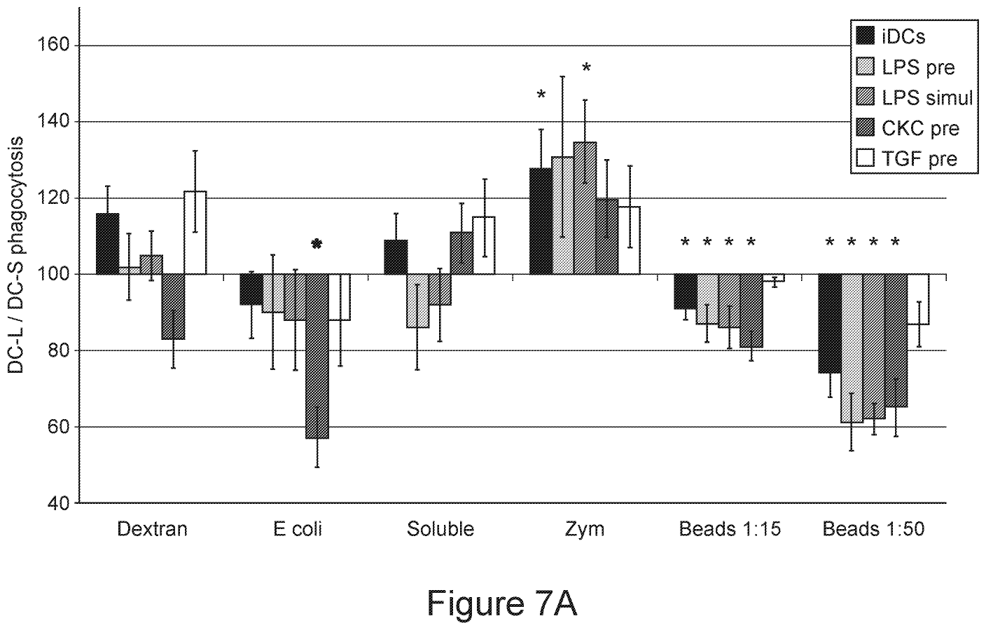

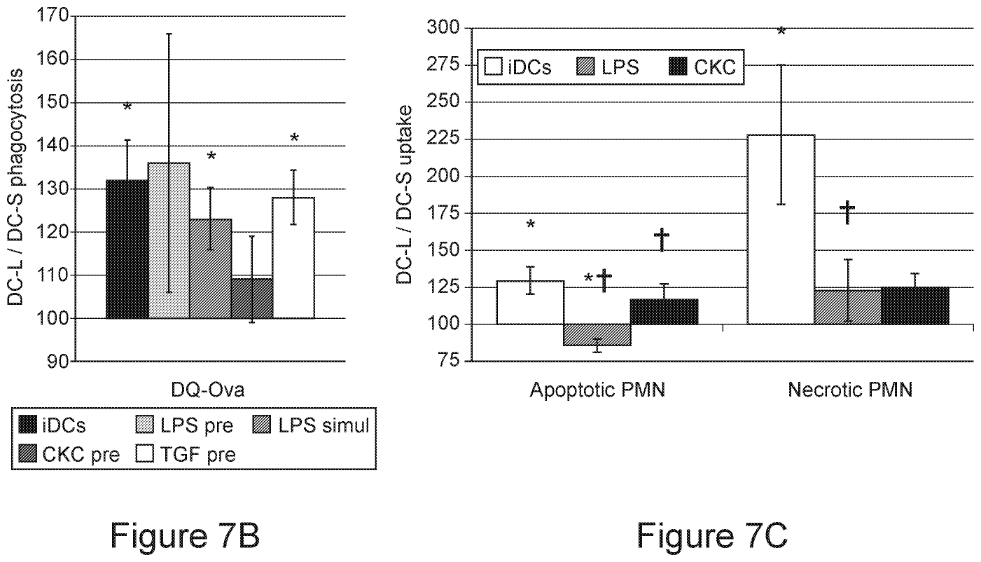

To continue exploring the functional differences between DC-S and DC-L, they were offered a variety of fluorescent targets at the immature stage; after stimulation with LPS, CKC, or TGF-β; or simultaneously with LPS. All fluorochromes used are insensitive to endosome acidification. As seen in FIG. 7A, DC-S and DC-L do not differ significantly in their capacity for dextran phagocytosis or the pinocytosis of a soluble dye. DC-S show a trend towards better phagocytosis of E. coli, which becomes significant after maturation with CKC. DC-S also show a significantly better capacity for the phagocytosis of latex beads (except for TGF-β-treated DCs), which becomes more prominent with a higher load of beads. DC-L cells, in contrast, show a better capacity for uptake of zymosan particles, which becomes significant after maturation with LPS given simultaneously. These results clearly indicate that cell size does not dictate all cellular functions; “bigger” is not always “more”. FIG. 7B demonstrates that DC-L show a stronger signal after being offered DQ™-ovalbumin (a self-quenched conjugate of ovalbumin that exhibits bright green fluorescence upon proteolytic degradation), an assay for antigen uptake and processing. Since both subsets perform pinocytosis similarly (FIG. 7A), and since the difference in expression of CD206 (which is a receptor of ovalbumin) is of significantly lower magnitude (FIG. 3), this suggests that DC-L is specifically better at antigen processing.

The DCs were next given fluorescently-labeled apoptotic polymorphonuclear (PMN) cells, either at the immature stage or after maturation with LPS or CKC. FIG. 7C shows that, even though the differences are not large, DC-L are better at the uptake of apoptotic PMN in the immature stage and after maturation with CKC, while DC-S are better at uptaking apoptotic PMN after maturation with LPS. The DCs were also offered necrotic PMN; surprisingly, uptake by DC-L surpassed uptake of apoptotic cells and was much more efficient than the rates observed for DC-S (FIG. 7C).

### Example 6. DC-L Acquires a Tolerogenic Phenotype after Uptake of Apoptotic Cells

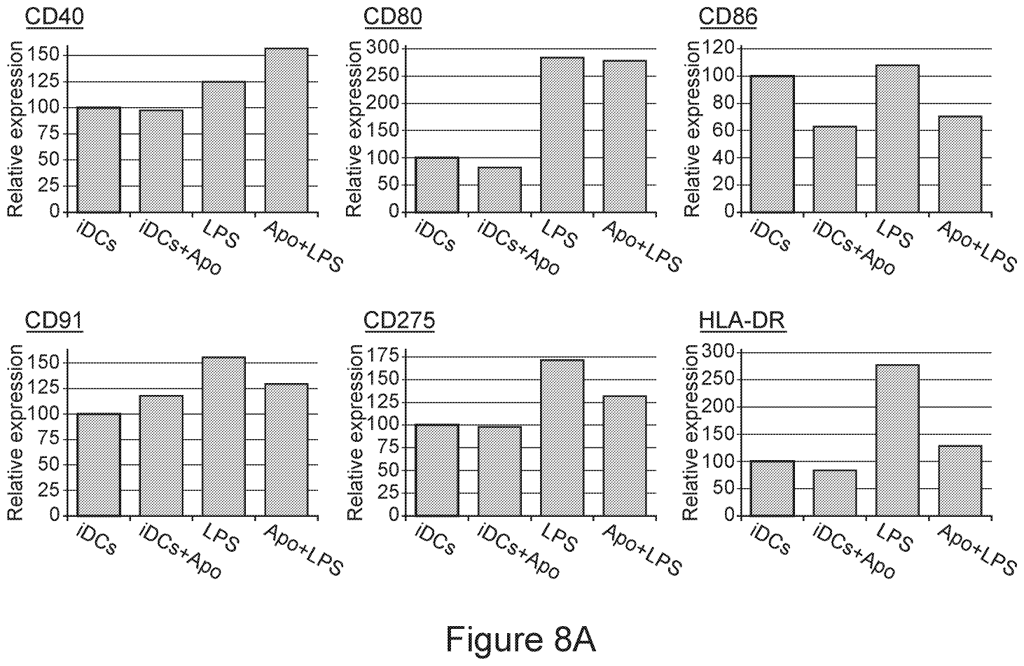

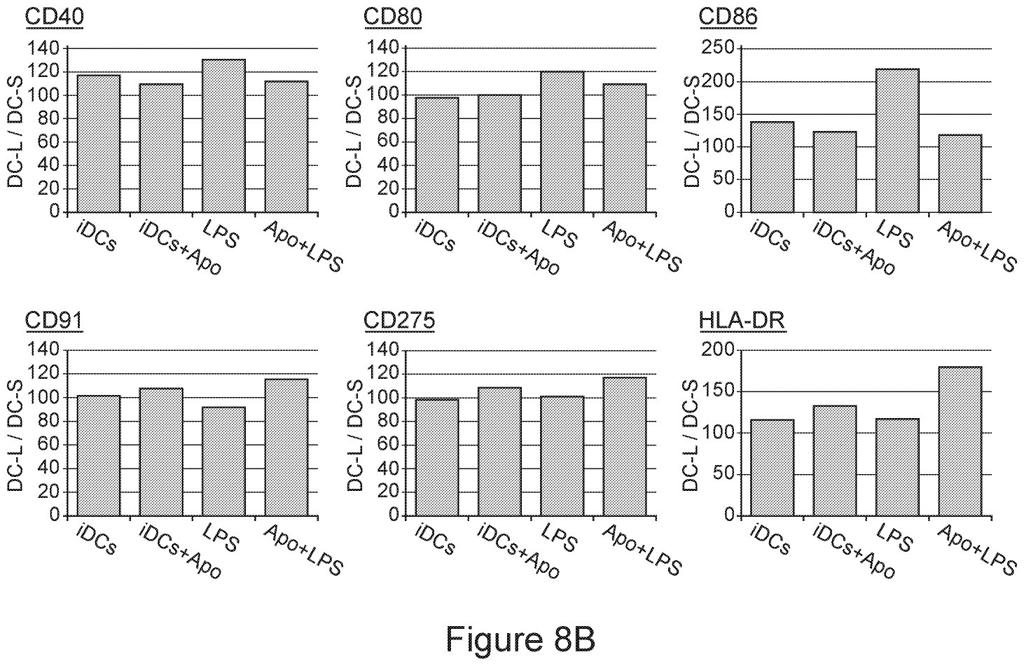

The differences between DC-S and DC-L following interaction with apoptotic cells were then assayed. Apoptotic peripheral blood mononuclear cells (PBMC) were added to iDCs at a ratio of 4:1 for 24 hours, with or without the addition of LPS 6 hours later. As shown at FIG. 8A, analysis of data for both sets of DCs reveals strong immunomodulatory effects from the apoptotic cells, with induction of a tolerogenic phenotype at the immature stage and inhibition of the response to LPS (except for CD40).

Analysis of DC-S vs DC-L (FIG. 8B) shows that after interaction of iDCs with apoptotic cells, the DC-L/DC-S expression ratio was reduced for CD40 and CD86, indicating a decrease of DC-L expression relative to DC-S. Concomitantly, the DC-L/DC-S ratio was increased for CD91, CD275, and, notably, HLA-DR. This pattern was repeated when LPS was added to DCs that had received apoptotic cells, but this time with an even increased magnitude. In order to confirm these results, these experiments were repeated using apoptotic PMN instead of PBMC, with similar results. Of note, these results show opposite responses of DC-S vs DC-L to LPS as compared to what was found when stimulating them without apoptotic cells (FIG. 3).

In summary, apoptotic cells induce an immune-suppressing phenotype among DC-S and DC-L, even after the addition of LPS. Moreover, following interaction with apoptotic cells, DC-L preferentially suppress costimulatory molecules while increasing their relative expression of HLA-DR, a trend that actually increased after addition of LPS to the apoptotic cells.

### Example 7. Production of Anti-CD19 CAR-T Cells

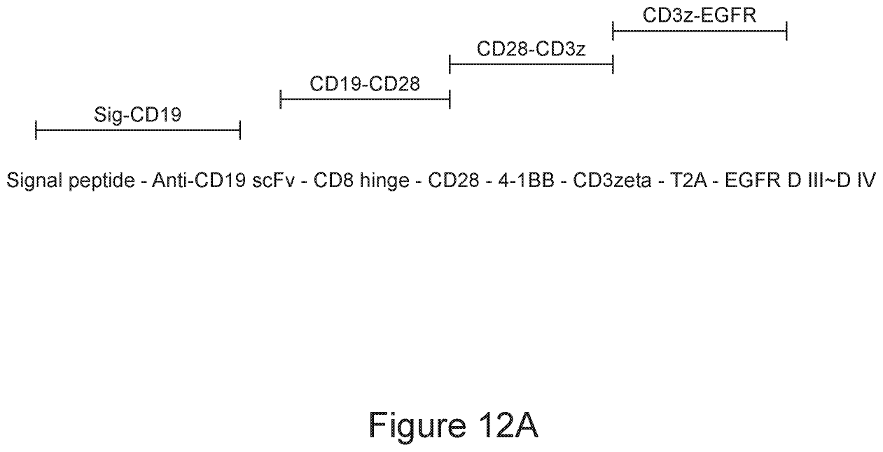

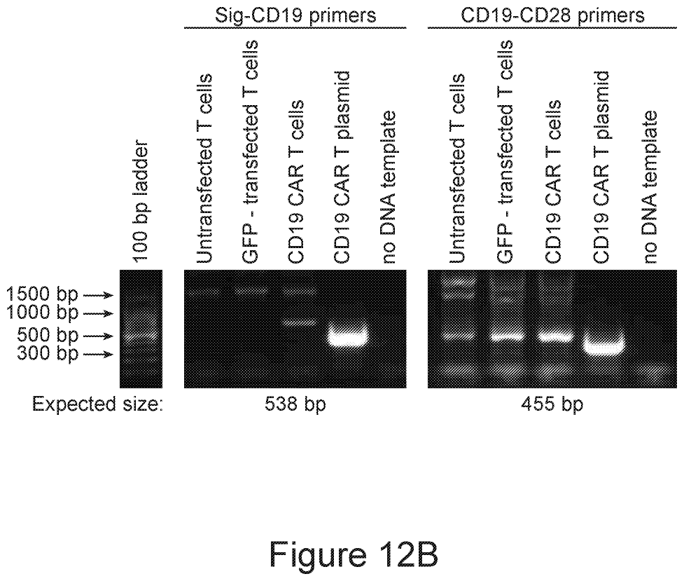

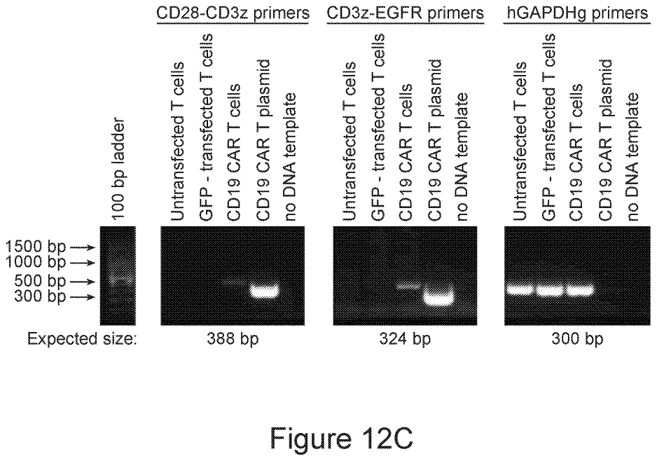

Mononuclear cells (MNC) were collected from buffy coat and frozen by DLI-like method. Upon thawing, MNC were activated by anti-CD3/CD28 beads (Miltenyi) at a cell-to-bead ratio of 1:3, respectively, for 48 hours. Virions were produced from Lenti-X 293 cell line (Clontech-Takara) transfected with Lenti-3^{rd} generation anti-CD19 CAR plasmid (Creative Biolabs; FIG. 12A) and pHelp1-3 packaging plasmids. The CAR comprises scFv of an anti-CD19 antibody linked to 4-1BB (CD137) and CD3ζ signaling domains Activated cells were infected by addition of 0.7 ml of the viral supernatant and 2 μg/ml polybrene (Chemicon) and centrifugation (500 g, 45 minutes, 32° C.). Cells were then incubated in the presence of 100 u/ml IL-2 (Peprotech). RT PCR was used (FIG. 12B and FIG. 12C) for detection of the successful transfection.

### Example 8. Production of Anti-CD19 CAR-DC and Cytotoxicity Assay

DC-L and DC-S population are transfected with the Lenti-3^{rd} generation anti-CD19 CAR plasmid, essentially as described in Example 7. Target cells (leukemia/lymphoma) and control cells are mixed in equal numbers in suspension, and each is pre-labeled with a different color (either CFSE or CMTMR). CAR-DC cells are then added and the cultures are incubated for 4 hours. After incubation, cells are stained with 7AAD (a viability marker, correlates well with PI), and cells are analyzed by FACS. The gating is done on 7AAD-negative (viable) cells, and calculations are done to deduce the % cytotoxicity of the CAR-DC cells.

### Example 9. Production of Bi-Specific CAR-DC

The genetic engineering of DC cells provides a means to rapidly generate anti-tumor DC cells for any tumor-associated antigen. This approach is used to produce immature DC-L (iDC-L), mature DC-L (mDC-L), immature DC-S (iDC-S) and mature DC-S (mDC-S) according to the present invention having an exogenous CAR, and to target these cell populations to specific cancer-related cells.

To obtain such CAR-DC cells, mature or immature, DC-L or DC-S cells according to the present invention are transfected with a plasmid carrying a gene encoding a tumor-associated-antigen-recognizing CAR, according to protocols well known in the field. Later, these cells are treated such that stable clones are produced, in which the CAR is stably expressed, e.g. a CAR for binding CD19, such as described in Example 7.

As selective pressure imparted by CAR-DC cells may yield antigen escape variants, the cells are optionally further transfected such that they stably expressed a plurality (2, 3, etc.) of different CARs, such that their targeting to cancer cells is made more specific, and less prone to the formation of antigen escape variants. For example, dually-targeted CAR-DCs may express both a CAR for binding CD19 and a CAR for binding CD22. In cytotoxicity experiments using CD19-CAR-DC cells, only target cells expressing human CD19 are lysed, as a function of Effector/Target ratio. In this experiment, Raji cells are used as the target cell population, as they are from a human B cell lymphoma, bearing both CD19 and CD20, and grow in suspension. To mimic the specific cell killing of the Raji cells, rituximab (Mabthera, Roche, Basel, Switzerland), a chimeric anti-human CD20 drug, is used. As negative control target cells, THP-1 cells, also growing in suspension, and not bearing CD19 or CD20, are used.

### Example 10. Use of CAR-DC in Treatment of Acute Lymphoid Leukemia (ALL)

Cancer cells obtained from acute lymphoid leukemia (ALL) patient are tested for extra-cellular marker expression profile. From this profile, one or more cancer-associated-antigens are identified. Then, CAR-DC cells, e.g. such as produced by the methods described in Example 7, are produced, the CAR(s) specifically recognizing the cancer-associated-antigens, e.g. CD19 and CD22.

Next, the patient is treated with the CAR-DC cells according to the present invention, when either each cell expressing one type of CAR (e.g. anti-CD19 or anti-CD22), or certain cells expressing an array of CARs, tailored to bind to a multiplicity of cancer-associated-antigens identified (e.g. anti-CD19 and anti-CD22). Dosing and administration regime may be determined on a case-by-case basis.

### REFERENCES

Tisch R. Immunogenic versus tolerogenic dendritic cells: a matter of maturation. Int. Rev. Immunol. 2010; 29(2):111-8.
Liu K, Nussenzweig M C. Origin and development of dendritic cells Immunol Rev. 2010; 234(1):45-54.
Mildner A, Jung S. Development and function of dendritic cell subsets. Immunity. 2014; 40(5):642-56.
Schlitzer A, Ginhoux F. Organization of the mouse and human DC network. Current opinion in immunology. 2014; 26:90-9.
Merad M, Sathe P, Helft J, Miller J, Mortha A. The dendritic cell lineage: ontogeny and function of dendritic cells and their subsets in the steady state and the inflamed setting. Annual review of immunology. 2013; 31:563-604.
Mildner A, Yona S, Jung S. A close encounter of the third kind: monocyte-derived cells. Advances in immunology. 2013; 120:69-103.
Collin M, McGovern N, Haniffa M. Human dendritic cell subsets. Immunology. 2013; 140(1):22-30.
Boltjes A, van Wijk F. Human dendritic cell functional specialization in steady-state and inflammation. Frontiers in immunology. 2014; 5:131.
Palucka K, Banchereau J. Dendritic-cell-based therapeutic cancer vaccines. Immunity. 2013; 39(1):38-48.
Duperrier K, Eljaafari A, Dezutter-Dambuyant C, Bardin C, Jacquet C, Yoneda K, et al. Distinct subsets of dendritic cells resembling dermal DCs can be generated in vitro from monocytes, in the presence of different serum supplements. J Immunol Methods. 2000; 238(1-2):119-31.
Chang C C, Wright A, Punnonen J. Monocyte-derived CD1a+ and CD1a− dendritic cell subsets differ in their cytokine production profiles, susceptibilities to transfection, and capacities to direct Th cell differentiation. J Immunol. 2000; 165(7):3584-91.
Xia C Q, Kao K J. Heparin induces differentiation of CD1a+ dendritic cells from monocytes: phenotypic and functional characterization. J Immunol. 2002; 168(3):1131-8.
Nunez R, Garay N, Bruno A, Villafane C, Bruno E, Filgueira L. Functional and structural characterization of two populations of human monocyte-derived dendritic cells. Exp Mol Pathol. 2004; 77(2):104-15.
Sondergaard J N, Brix S. Isolation of IL-12p70-competent human monocyte-derived dendritic cells. J Immunol Methods. 2012; 386(1-2):112-6.
Liu K, Victora G D, Schwickert T A, Guermonprez P, Meredith M M, Yao K, et al. In vivo analysis of dendritic cell development and homeostasis. Science. 2009; 324(5925):392-7.
Geissmann F, Jung S, Littman D R. Blood monocytes consist of two principal subsets with distinct migratory properties. Immunity. 2003; 19(1):71-82.
Varol C, Vallon-Eberhard A, Elinav E, Aychek T, Shapira Y, Luche H, et al. Intestinal lamina propria dendritic cell subsets have different origin and functions. Immunity. 2009; 31 (3):502-12.
Dominguez P M, Ardavin C. Differentiation and function of mouse monocyte-derived dendritic cells in steady state and inflammation. Immunol Rev. 2010; 234(1):90-104.
Auffray C, Sieweke M H, Geissmann F. Blood monocytes: development, heterogeneity, and relationship with dendritic cells. Annual review of immunology. 2009; 27:669-92.
Alonso M N, Wong M T, Zhang A L, Winer D, Suhoski M M, Tolentino L L, et al. T(H)1, T(H)2, and T(H)17 cells instruct monocytes to differentiate into specialized dendritic cell subsets. Blood. 2011; 118(12):3311-20.
Hammer G E, Ma A. Molecular control of steady-state dendritic cell maturation and immune homeostasis. Annual review of immunology. 2013; 31:743-91.
Green D R, Ferguson T, Zitvogel L, Kroemer G Immunogenic and tolerogenic cell death. Nature reviews. 2009; 9(5):353-63.
Sancho D, Joffre O P, Keller A M, Rogers N C, Martinez D, Hernanz-Falcon P, et al. Identification of a dendritic cell receptor that couples sensing of necrosis to immunity. Nature. 2009; 458(7240):899-903.
Stein G M, Pfuller U, Schietzel M, Bussing A. Expression of interleukin-4 in apoptotic cells: stimulation of the type-2 cytokine by different toxins in human peripheral blood mononuclear and tumor cells. Cytometry. 2000; 41(4):261-70.
Krispin A, Bledi Y, Atallah M, Trahtemberg U, Verbovetski I, Nahari E, et al. Apoptotic cell thrombospondin-1 and heparin-binding domain lead to dendritic-cell phagocytic and tolerizing states. Blood. 2006; 108(10):3580-9.
Stranges P B, Watson J, Cooper C J, Choisy-Rossi C M, Stonebraker A C, Beighton R A, et al Elimination of antigen-presenting cells and autoreactive T cells by Fas contributes to prevention of autoimmunity. Immunity. 2007; 26(5):629-41.
Kushwah R, Hu J. Dendritic cell apoptosis: regulation of tolerance versus immunity. J Immunol. 2010; 185(2):795-802.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-40. (canceled)

41. A cell preparation of a substantially pure human monocyte-derived dendritic cell (mdDC) population, selected from the group consisting of:
a) DC-Large (DC-L), characterized, based on their mean size, granularity and membrane complexity, respectively, as size^{high}, gran^{high}, complexity^{high}; and
b) DC-Small (DC-S), characterized based on their mean size, granularity and membrane complexity, respectively as size^{low}, gran^{low}, complexity^{low}.

42. The cell preparation of claim 41, wherein the human mdDC population is a population of immature mdDC or wherein the human mdDC population is a population of mature mdDC.

43. The cell preparation of claim 41, wherein said cells are selected from the group consisting of:
i. immature DC-L (iDC-L), further characterized by their expression levels of surface markers as CD11c^{high}, CD47^{high}, and DCSIGN^{high},
ii. immature DC-S (iDC-S), further characterized by their expression levels of surface markers as CD11c^{low}, CD47^{low}, and DCSIGN^{low},
iii. mature DC-L (mDC-L), produced by incubating a population of iDC-L ex-vivo with at least one maturation signal comprising lipopolysaccharide (LPS), zymosan, PgE₂, tumor necrosis factor α (TNF-α), interleukin 1β (IL-1β), transforming growth factor β (TGF-β), or combinations thereof, and
iv. mature DC-S (mDC-S), produced by incubating a population of iDC-S ex-vivo with at least one maturation signal comprising LPS, zymosan, PgE2, TNF-α, IL-1β, TGF-β, and combinations thereof.

44. The cell preparation of claim 41, wherein said cell population has been generated by a method comprising a) providing a population of human mdDC by ex-vivo differentiation of monocytes in the presence of granulocyte-macrophage colony-stimulating factor (GM-CSF) and IL-4, and b) isolating said cell population using cell sorting.

45. A cell vaccine or an immuno-modulating cell composition, comprising: the cell preparation of claim 41, and/or a T cell preparation activated in the presence of the cell preparation of claim 41.

46. The cell vaccine of claim 45, wherein said mdDC population has been genetically modified to express at least one targetor, co-stimulatory molecule and/or antigen, wherein said at least one targetor comprises at least one chimeric antigen receptor (CAR).

47. The cell vaccine of claim 45, comprising: a cell preparation as defined in claim 1, pulsed with at least one disease-associated antigen, said cell vaccine further comprising a pharmaceutically acceptable carrier, excipient and/or adjuvant.

48. The cell vaccine of claim 47, wherein said human mdDC population is a population of mature DC-L, obtained by ex-vivo incubation of iDC-L in the presence of the at least one disease-associated antigen and at least one maturation signal, wherein said disease-associated antigen is implicated in the etiology and/or pathology of cancer or of an infective disease associated with a viral, bacterial, fungal or parasitic infection.

49. The cell vaccine of claim 48, wherein said disease-associated antigen is a tumor-associated antigen selected from the group consisting of B7H3, CAIX, CD44 v6/v7, CD171, CEA, EGFRvIII, EGP2, EGP40, EphA2, and ErbB2(HER2), or a viral antigen associated with a Cytomegalovirus (CMV), Epstein Barr Virus (EBV), Human Immunodeficiency Virus (HIV), or influenza virus.

50. The cell vaccine of claim 48, wherein said mdDC population has been genetically modified to express at least one CAR that specifically binds a cell-surface tumor-associated antigen presented on a cancer cell, and wherein the cancer is selected from the group consisting of melanoma, urinary tract cancer, gynecological cancer, he ad and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers.

51. The cell vaccine of claim 50, wherein the cancer is acute lymphoid leukemia, and wherein said cell population expresses at least one CAR that specifically binds to CD19 and/or at least one CAR that specifically binds to CD22.

52. The immuno-modulating cell composition of claim 45, comprising: a cell preparation of a substantially pure human monocyte-derived dendritic cell (mdDC) population, selected from the group consisting of:
a) DC-Large (DC-L), characterized, based on their mean size, granularity and membrane complexity, respectively, as size^{high}, gran^{high}, complexity^{high}; and
b) DC-Small (DC-S), characterized based on their mean size, granularity and membrane complexity, respectively as size^{low}, gran^{low}, complexity^{low},
pulsed with at least one disease-associated antigen implicated in the etiology and/or pathology of an autoimmune or inflammatory disease and/or with necrotic or apoptotic cells, said cell composition further comprising a pharmaceutically acceptable carrier, excipient and/or adjuvant.

53. The immuno-modulating cell composition of claim 52, wherein said human mdDC population is selected from the group consisting of:
i. a population of mature DC-S obtained by ex-vivo incubation of iDC-S in the presence of the at least one antigen implicated in the etiology and/or pathology of an autoimmune or inflammatory disease and with at least one maturation signal, and
ii. a population of mature DC-L obtained by ex-vivo incubation of iDC-L in the presence of necrotic or apoptotic cells and with at least one maturation signal,
and wherein said antigen is implicated in the etiology or pathology of a T cell mediated disease selected from the group consisting of autoimmune diseases, chronic non-resolving inflammatory diseases, and graft rejection.

54. A method for the treatment or amelioration of cancer or an infective disease in a subject in need thereof, comprising administering to the subject an effective amount of the cell vaccine of claim 45.

55. A method for inducing or enhancing an immunogenic reaction towards antigens implicated in the etiology and/or pathology of cancer or an infective disease in a subject in need thereof, comprising administering to the subject an effective amount of the cell vaccine of claim 47.

56. The method of claim 55, wherein said antigen is a tumor-associated antigen and wherein said tumor is selected from the group consisting of melanoma, urinary tract cancer, gynecological cancer, head and neck carcinoma, primary brain tumor, bladder cancer, liver cancer, lung cancer, breast cancer, ovarian cancer, prostate cancer, cervical cancer, colon cancer and, cancer of the intestinal tract, bone malignancies, connective and soft tissue tumors, skin cancers and hematopoietic cancers.

57. A method for the treatment or amelioration of an autoimmune or an inflammatory disease in a subject in need thereof, comprising administering to the subject an effective amount of the immuno-modulating cell composition of claim 52.

58. A method for induction of a tolerogenic immune reaction towards antigens implicated in the etiology and/or pathology of an autoimmune or inflammatory disease in a subject in need thereof, comprising administering to the subject an effective amount of the immuno-modulating cell composition of claim 52.

59. The method of claim 58, wherein said antigen is implicated in the etiology or pathology of a T cell mediated disease selected from the group consisting of: autoimmune diseases, chronic non-resolving inflammatory diseases, and graft rejection.

60. The method of claim 59, wherein said autoimmune disease is selected from the group consisting of multiple sclerosis, rheumatoid arthritis, juvenile rheumatoid arthritis, autoimmune neuritis, systemic lupus erythematosus, psoriasis, Type I diabetes, Sjogren's disease, thyroid disease, myasthenia gravis, sarcoidosis, autoimmune uveitis, inflammatory bowel disease and autoimmune hepatitis.

61. An ex-vivo method for generating the cell preparation of claim 41, comprising a) providing a population of human mdDC by ex-vivo differentiation of monocytes in the presence of granulocyte-macrophage colony-stimulating factor (GM-CSF) and IL-4, and b) isolating said cell population using cell sorting.

62. The method of claim 61, wherein said cell population is isolated by a method comprising cell sorting based on at least one parameter selected from the group consisting of cell size, cell granularity, membrane complexity and the level of surface marker expression, wherein said surface marker comprises a plurality of markers selected from the group consisting of: αVβ5, CD11c, CD47, CD36, CD274 (PDL1), CD11b (CR3), CD6 (B7.2), CD85k (ILT3), CD40, CD324 (E cadherin), CD45, HLA-DR, TLR-1, CD33 (SIGLEC-3), CD266 (TWEAK-R), CD206, DCSIGN, CD200 (OX2), CD172a (SIRPα), CD273 (PDL2), CD141, CCR5, HLA-ABC, CD85j (ILT2), CD54 (ICAM-1), CD80 (B7.1), CD16 (FcγRIII), FcεRI, CD275 (ICOS-L), and CD25 (IL2R).

63. The method of claim 62, wherein steps a) and b) are performed without the addition of exogenous activation or maturation stimuli, or wherein the method further comprises genetically modifying said mdDC population to express at least one CAR, or wherein the method further comprises incubating said cells ex-vivo in the presence of the at least one disease-associated antigen and at least one maturation signal comprising LPS, zymosan, PgE₂, TNF-α, IL-1β, TGF-β, or combinations thereof.
